(19) United States
(12) Patent Application Publication
(10) Pub. No.: US 2013/0331643 A1
(43) Pub. Date: Dec. 12, 2013

(54) SELF-ADJUSTING GASTRIC BAND

(71) Applicant: Allergan, Inc., Irvine, CA (US)

(72) Inventors: Sean Snow, Carpinteria, CA (US); Mitchell H. Babkes, Santa Clarita, CA (US); Marcos Borrell, Goleta, CA (US); Christopher Deuel, Somerville, MA (US); Zachary Dominguez, Santa Barbara, CA (US); Ethan Franklin, Goleta, CA (US); Babak Honaryar, Orinda, CA (US); Erik Torjesen, Goleta, CA (US)

(73) Assignee: Allergan, Inc., Irvine, CA (US)

(21) Appl. No.: 13/965,041

(22) Filed: Aug. 12, 2013

Related U.S. Application Data
(63) Continuation of application No. 13/149,585, filed on May 31, 2011, now abandoned.
Application No. 13/149,585 is a continuation-in-part of application No. 12/770,617, filed on Apr. 29, 2010.

(51) Int. Cl. A61F 5/00

(52) U.S. Cl. CPC A61F 5/005
USPC 600/37

(57) ABSTRACT

Generally described herein are automatic, self-adjusting, gastric banding systems and improvements thereof, that are capable of automatically relaxing and contracting in response to a large bolus passing through the area of a patient's stomach constricted by a gastric band. Alternatively, and/or in addition in one or more embodiments, the gastric banding systems described herein may also help prevent pouch dilatation and/or erosion. The apparatus and systems described herein aid in facilitating obesity control and/or treating obesity-related diseases while generally being non-invasive once implanted. Furthermore, certain embodiments of the self-adjusting gastric banding systems disclosed herein are automatically adjustable without complicated fluid control mechanisms, flow rate limiting devices, and/or valves. The automatic adjustments may also be made in response to other changes in the patient's esophageal-gastric junction, for example, in response to size, shape, and or location changes.

### CROSS REFERENCE TO RELATED APPLICATIONS

This application is a continuation of U.S. patent application Ser. No. 13/149,585, filed May 31, 2011, which is a continuation-in-part of U.S. patent application Ser. No. 12/770,617 filed on Apr. 29, 2010, the entire disclosure of which is incorporated herein by this specific reference.

### FIELD

The present invention generally relates to medical systems, devices and uses thereof for treating obesity and/or obesity-related diseases. More specifically, the present invention relates to gastric banding systems that may self-adjust to changes in a patient.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach may provide a feeling of satiety or fullness that discourages overeating. Unlike gastric bypass procedures, gastric band apparatus are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

However, the level of tightness of the gastric band may effect the patient's sensations and satisfaction level. In other words, if the gastric band is underfilled, the patient may experience hunger; but if the gastric band is overfilled (and thus “too tight”), the patient may experience tightness in the chest region, suffer from a food bolus blockage, and the like.

Sometimes, adjustment of a gastric band may be desirable in between adjustments made by a physician. For example, during normal operation of the gastric band, the band applies pressure to the outer surface of the upper stomach. However, it may be difficult to achieve the most effective level of tightness, and further, physicians may tend to err on the side of underfilling the gastric band (thereby decreasing efficacy of the gastric band system) as they consider the risk of overfilling the gastric band.

Some attempts have been made to develop a gastric band that promotes an effective fill level. For example, with reference to FIGS. 1A-1B, Lau, et al., U.S. Patent Pub. No. 2010/0191271 discloses an elastic bladder that is in constant fluid communication with the expandable balloon portion of a gastric band in order to continuously adjust the gastric band. With reference to FIG. 1C, Lau, et al., U.S. Patent Pub. No. 2010/0191265 discloses an alternative elastic bladder having four wings.

With reference to FIG. 2A, Coe, et al., U.S. Patent Pub. No. 2009/0216255 discloses a flow control device A that moves fluid between a hydraulic restriction system and a fluid source B. The additional flow control device A controls a rate of fluid flow between the restriction device and the fluid source B. With reference to FIG. 2B, Coe, et al., European Patent Application No. 2 074 970 A1 discloses a separate restriction device and pressure adjustment device C. The pressure adjustment device C regulates a constant force applied by the restriction device using, for example, a bellows and a spring.

With reference to FIG. 2C, Lechner, U.S. Patent Pub. No. 2009/0054914 discloses a controllable stomach band that has a chamber for controlling restriction of the stomach band. The chamber is coupled to a separate pressure chamber D that receives fluid leaving the chamber in the stomach band. The pressure chamber D is separated from the esophageal-gastric junction of the patient's stomach.

Further, with respect to FIG. 3, Steffen, U.S. Patent Pub. No. 2009/0062826 discloses an adjustable gastric band with a “conveyance device” that is powered by a “power storage device.” The power storage device operates the conveyance device to move fluid between expandable chambers to adjust the gastric band.

Accordingly, it is desirable to develop a self-adjusting gastric band that will provide the needed pressure to the stomach to create the stoma and facilitate weight control, but that will also adapt and open up to allow a large bolus to pass through. It is further desirable to create an automatically self-adjusting gastric band that does not require an electrical power source and/or external adjustments, to allow a large bolus to pass through.

Additionally, it is desirable to make the adjustments without additional, complicated fluid control mechanisms, flow rate limiting devices, and/or valves to regulate the transfer of fluid within the self-adjusting gastric band. Moreover, it is desirable to make these adjustments to the gastric band utilizing compliant components to both reduce and restore the constriction of the gastric band.

### SUMMARY

This Summary is included to introduce, in an abbreviated form, various topics to be elaborated upon below in the Detailed Description.

Generally described herein are automatic, self-adjusting, gastric banding systems and improvements thereof that are capable of automatically relaxing and contracting in response to a large bolus passing through the area of a patient's stomach constricted by a gastric band. Alternatively, and/or in addition in one or more embodiments, the gastric banding systems described herein may also help prevent pouch dilatation and/or erosion. The apparatus and systems described herein may aid in facilitating obesity control and/or treating obesity-related diseases while generally being non-invasive once implanted. The automatic adjustments may also be made in response to other changes in the patient's esophageal-gastric junction, for example, in response to size, shape, and/or location changes.

In one embodiment, a self-adjusting gastric banding system for the treatment of obesity that adjusts to allow a bolus to pass through a constriction in a patient's stomach formed by the self-adjusting gastric banding system is disclosed. The self-adjusting gastric banding system comprises a gastric band having an inflatable portion disposed about the stomach of the patient, the inflatable portion capable of constricting the stomach, an access port fluidly coupled to the inflatable portion, the access port capable of receiving a fluid to inflate, or removing a fluid to deflate, the inflatable portion of the gastric band, a tubing coupled to the gastric band and the access port, the tubing carrying fluid between the inflatable portion of the gastric band and the access port, and a compliant reservoir attached to at least a portion of the tubing and configured to receive fluid from the inflatable portion of the gastric band for automatically relaxing the constriction formed by the inflatable portion of the gastric band sufficiently to allow the bolus to pass through the constriction.

Further, the gastric band comprises a first compliant portion coupled to a part of the system. For example, the first compliant portion may be coupled to the inflatable portion, the access port, and/or the tubing. The first compliant portion automatically relaxes the constriction formed by the self-adjusting gastric band and allows the large bolus to pass through the constriction. After the bolus passes through the constriction, the gastric band automatically returns to its previous state.

In accordance with various embodiments, the first compliant portion facilitates automatically relaxing the constriction formed by the self-adjusting gastric band without causing a fluid to exit the inflatable portion of the gastric band. For example, the self-adjusting gastric band may comprise a ring coupled to the inflatable portion of the gastric band. The ring provides structure and support to the inflatable portion, and the ring facilitates disposing the inflatable portion about the esophageal-gastric junction.

The ring may be a flexible ring with a diameter that expands when a predetermined pressure is generated in the inflatable portion. For example, the predetermined pressure may be generated in response to the large bolus passing through the esophageal-gastric junction. The flexible ring expands to automatically relax the constriction formed by the self-adjusting gastric band. In various embodiments, the ring has a durometer in the range of approximately 20 to approximately 70.

According to a further embodiment, the first compliant portion receives a first amount of fluid from the inflatable portion when the large bolus causes a pressure in the first compliant portion to exceed an expansion pressure. Receiving the first amount of fluid from the inflatable portion facilitates relaxing the constriction formed by the self-adjusting gastric band and allowing the large bolus to pass through the constriction.

In an embodiment, the first compliant portion is fluidly coupled to the inflatable portion. The first compliant portion facilitates removing the first amount of fluid from the inflatable portion when the large bolus passes through the constriction.

According to another embodiment, the self-adjusting gastric band further comprises a second compliant portion fluidly coupled to the access port. The second compliant portion automatically removes a second amount of fluid from the inflatable portion via the access port to facilitate relaxing the constriction formed by the inflatable portion.

The tubing of the gastric banding system may be compliant tubing that expands in response to a pressure in the tubing exceeding a tubing expansion pressure when the large bolus passes through the constriction formed by the self-adjusting gastric band. In this regard, a third amount of fluid is removed from the inflatable portion when the compliant tubing expands. The tubing may be perforated to facilitate receiving the fluid from the inflatable portion via the tubing.

Further, another embodiment of the self-adjusting gastric band comprises a third compliant portion fluidly coupled to the tubing for automatically receiving a third amount of fluid from the inflatable portion via the tubing when the large bolus enters the esophageal-gastric junction. Receiving the third amount of fluid from the inflatable portion facilitates relaxing the constriction formed by the gastric band and allowing the large bolus to pass through the constriction.

The compliant components, according to various embodiments, comprise a kink-resisting feature. Further, the compliant components may comprise a leak-resisting feature. These components may be an elastic polymer, a balloon, a rubber container, a silicone container, a collapsible container, a bellows, and combinations thereof.

In an embodiment, a vacuum device may be used in transferring fluid from a gastric band to a reservoir to assist the patient in facilitating the passage of a large bolus through a constriction of the gastric band.

In an embodiment, a gastric banding system may include a tube with a gap or cut located within a balloon or reservoir. The gap or cut may allow for fluid transfer between fluidly-coupled components to the balloon or reservoir. In this manner, fluid may flow to the reservoir from an inflatable portion of the gastric banding system to relieve the pressure induced by the large bolus.

In an embodiment, a gastric banding system may include a tube with slits or holes located within a balloon or reservoir. The slits or holes may allow for fluid transfer between fluidly-coupled components to the balloon or reservoir. In this manner, fluid may flow to the reservoir from an inflatable portion of the gastric banding system to relieve the pressure induced by the large bolus.

In an embodiment, a gastric banding system may include a tube-like reservoir configured to inflate and deflate based on a volume level within the reservoir. The tube-like reservoir may be compliant and may have a star-shaped outer circumference in a first state and a circular, uniform outer circumference in a second state. Alternatively, or in addition, the middle portions of the tube-like reservoir may expand in diameter as more fluid is added.

In an embodiment, a gastric banding system may include a flattened reservoir having a uniform configuration, an indented configuration or a u-shaped configuration for inflating and deflating based on a volume level within the reservoir. In addition to providing a unique, expandable shape profiled over a continuous length, improved performance may be achieved through reduction of the effects of the external forces on fluid within an adjacent, non-compliant component.

In an embodiment, a gastric banding system may include a tube-on-tube reservoir having an outer non-compliant tube intended to prevent kinking, bending, or any other fluid disruption to an inner compliant tube. The inner compliant tube may be separated from the outer non-compliant tube by a gap which allows the inner compliant tube to expand (to fill the gap).

In an embodiment, a gastric banding system may include a reservoir with a winged portion, a coiled portion or an enlarged portion configured to inflate and deflate based on a volume level within the reservoir. These reservoirs may be attached or coupled to an access port of a gastric banding system.

In an embodiment, a gastric banding system may include a reservoir with internal structures such as a spring, a cage or a ring. The internal structures may act to prevent kinking, bending of the compliant portion, or otherwise prevent fluid flow interruptions within the reservoir.

In an embodiment, a gastric banding system may include a reservoir with external structures such as a skeleton or a protective layer. The external structures may act to prevent kinking, bending of the compliant portion, or otherwise prevent fluid flow interruptions within the reservoir.

In an embodiment, a gastric banding system may include a reservoir having depressions, pleatings or longitudinal structures along an outer circumferential perimeter. The depressions, pleatings or longitudinal structures may allow for easier and more predictable deflation of the reservoir, e.g., during implantation or removal procedures.

In an embodiment, a gastric banding system may have one or more reservoirs oriented radially from the gastric band.

In an embodiment, a gastric banding system may include non-saline fill substances such as a gel, a pseudoplastic material, or a Bingham plastic. The non-saline fill substances may have different properties that allow for different pressure behaviors when an external pressure is applied to the gastric banding system (e.g., when a large or small bolus is swallowed by the patient).

In an embodiment, a gastric banding system may be self-contained and may be filled with one or more of a various number of different fill substances. The self-contained gastric banding system may include a ring, one or more cushions, and one or more hinges that flex when a pressure is exerted on the cushions (e.g., when a patient swallows a large or small bolus of food).

In an embodiment, a gastric banding system may include a hybrid gas-saline component. The gas component may be a balloon or other gas filled member coated with a gas-impermeable coating and may be designed to flow between other components of the gastric banding system based on the pressure exerted, e.g., by a large bolus passing through a constriction formed by the gastric band.

In an embodiment, a gastric banding system may include an access port having a movable surface, which may move in response to a pressure change within the gastric banding system. For example, the movable surface may be a complaint portion that moves to increase the volume of the fluid portion (and therefore increases compliance) when the pressure increases within the gastric banding system (e.g., in response to a large bolus moving through a constriction of the gastric band). Once the pressure is reduced (e.g., the large bolus passing through the constriction), the movable surface may return to its original position, thereby decreasing the volume of the fluid portion of the access port. The access port may further include a septum, a fluid-permeable membrane, o-rings and a gas spring portion filled with a gas. Alternatively or in addition, the gas spring portion may be replaced with a wave spring, a cantilever spring, a constant force spring, a coil spring, a leaf spring, a Belleville spring, a hybrid polymer coil-air spring and the like.

In an embodiment, a gastric banding system may include a flow-rate control device. The flow rate control device may improve comfort of a patient when a large bolus is passing through the constriction by increasing the rate that fluid flows out of the gastric band and into the reservoir. In this manner, the pressure increase may be significantly reduced. Once the bolus passes, the reservoir-side pressure may gradually decrease as the gastric band begins to inflate again with fluid, and the gastric banding system may approach the equilibrium pressure.

In an embodiment, a gastric banding system may include a gastric band without a locking portion, and with a more inflexible ring portion. More particularly, instead of having a flexibly-stiff ring locked in place at an open end (as traditionally utilized in a standard gastric band), the gastric banding system does not include a locking portion and replaces the flexibly-stiff ring with a more inflexible ring such as a snap ring, a split ring, retaining ring and the like.

In an embodiment, a gastric banding system may include a ring and corresponding inflatable portions having a wider portion. The wider portion may operate to stimulate and restrict the patient's esophageal-gastric junction when the patient is not eating or swallowing small boluses. When the patient swallows medium-sized boluses, the wider portion may channel the medium bolus through the standard portions. And when the patient swallows large boluses, the wider portion may function to relieve the stress on the patient's tissue and assist to prevent formations of pouch dilatations.

In an embodiment, a gastric banding system may include a gastric band without a ring portion. The gastric band without a ring portion may be more flexible (e.g., by having decreased ring stiffness) than a standard gastric band, thereby resulting in a gastric banding system having muted pressure or force spikes on the tissues (e.g., in the esophageal-gastric junction) in a patient when the patient consumes a large bolus of food.

In an embodiment, a gastric banding system may include a gastric band with a modified ring portion. The modified ring portion may be more flexible (e.g., by having decreased ring stiffness) than a standard gastric band, thereby resulting in gastric banding systems having muted pressure or force spikes on the tissues (e.g., in the esophageal-gastric junction) in a patient when the patient consumes a large bolus of food.

### BRIEF DESCRIPTION OF THE DRAWINGS

The features, obstacles, and advantages of the present invention will become more apparent from the detailed description set forth below when taken in conjunction with the drawings, wherein:

FIG. 1A illustrates a prior art system that includes an elastic bladder.

FIG. 1B illustrates a prior art system that includes an elastic bladder having a fold.

FIG. 1C illustrates a prior art system that includes an elastic bladder having four wings.

FIG. 2A illustrates a prior art system that includes a flow rate limiting device.

FIG. 2B illustrates a prior art system that includes a fluid control mechanism.

FIG. 2C illustrates a prior art system that includes a valve and a chamber separated from the esophageal-gastric junction.

FIG. 3 illustrates a prior art system that includes a gastric band with a “conveyance device” that is powered by a “power storage device.”

FIG. 4 illustrates an exploded, perspective view of a self-adjusting gastric banding system according to an embodiment of the present invention.

FIG. 5 illustrates an exploded, perspective view of a self-adjusting gastric banding system having various compliant components according to an embodiment of the present invention.

FIG. 6 illustrates an exploded, perspective view of another self-adjusting gastric banding system having various compliant components according to an embodiment of the present invention.

FIG. 7A illustrates a chart showing pressure-volume curves for a standard gastric band and a self-adjusting gastric band according to an embodiment of the present invention.

FIG. 7B illustrates a chart showing pressure-time curves for a standard gastric band and a self-adjusting gastric band subject to a period of obstruction according to an embodiment of the present invention.

FIG. 8A illustrates a vacuum device configured to provide suction on a patient's skin proximal to the compliant reservoir according to an embodiment of the present invention.

FIG. 8B illustrates the vacuum device of FIG. 8A in an operational state according to an embodiment of the present invention.

FIG. 9 illustrates a cross-sectional view of a compliant tubing-reservoir system according to an embodiment of the present invention.

FIG. 10 illustrates a cross-sectional view of a compliant tubing-reservoir system having a gap or cut sealed within a reservoir according to an embodiment of the present invention.

FIG. 11 illustrates a cross-sectional view of a compliant tubing-reservoir system having slits or holes sealed within a reservoir according to an embodiment of the present invention.

FIG. 12 illustrates an exploded, perspective view of a compliant tubing-reservoir system according to an embodiment of the present invention.

FIG. 13A illustrates an exploded, perspective view of a compliant tubing-reservoir system having a tube-shaped reservoir according to an embodiment of the present invention.

FIG. 13B illustrates a cross-sectional view of a non-extruded area of the tube-shaped reservoir of FIG. 13A according to an embodiment of the present invention.

FIG. 13C illustrates a cross-sectional view of an as-extruded area of the tube-shaped reservoir of FIG. 13A according to an embodiment of the present invention.

FIG. 14A illustrates a perspective view of a tube-shaped reservoir in a non-expanded state according to an embodiment of the present invention.

FIG. 14B illustrates a perspective view of the tube-shaped reservoir of FIG. 14A in an as-expanded state according to an embodiment of the present invention.

FIG. 15 illustrates an exploded, perspective view of a compliant tubing-reservoir system having a tube-shaped reservoir according to an embodiment of the present invention.

FIG. 15A illustrates a cross-sectional view of the tube-shaped reservoir of FIG. 15 according to an embodiment of the present invention.

FIG. 16 illustrates a cross-sectional view of another tube-shaped reservoir according to an embodiment of the present invention.

FIG. 17A illustrates a cross-sectional view of a tube-shaped reservoir in a first state according to an embodiment of the present invention.

FIG. 17B illustrates a cross-sectional view of the tube-shaped reservoir of FIG. 17A in a second state according to an embodiment of the present invention.

FIG. 17C illustrates a cross-sectional view of the tube-shaped reservoir of FIG. 17A in a third state according to an embodiment of the present invention.

FIG. 18A illustrates a close-up view of a compliant tube-on-tube reservoir system according to an embodiment of the present invention.

FIG. 18B illustrates a cross-sectional view of the compliant tube-on-tube reservoir system of FIG. 18A according to an embodiment of the present invention.

FIG. 18C illustrates a partial, exploded view of the compliant tube-on-tube reservoir system of FIG. 18A according to an embodiment of the present invention.

FIG. 18D illustrates a partial, exploded view of the compliant tube-on-tube reservoir system of FIG. 18A according to an embodiment of the present invention.

FIG. 18E illustrates a cross-sectional view of the compliant tube-on-tube reservoir system of FIG. 18D according to an embodiment of the present invention.

FIG. 18F illustrates a perspective view of a portion of the compliant tube-on-tube reservoir system of FIG. 18A according to an embodiment of the present invention.

FIG. 18G illustrates a partial, exploded view of the compliant tube-on-tube reservoir system of FIG. 18A according to an embodiment of the present invention.

FIG. 19A illustrates a perspective view of a coiled reservoir in a first state according to an embodiment of the present invention.

FIG. 19B illustrates a perspective view of the coiled reservoir of FIG. 19A in a second state according to an embodiment of the present invention.

FIG. 20A illustrates a perspective view of a reservoir having winged portions in a first state according to an embodiment of the present invention.

FIG. 20B illustrates a perspective view of the reservoir having winged portions of FIG. 20A in a second state according to an embodiment of the present invention.

FIG. 21A illustrates a perspective view of a tube-shaped reservoir having a closed end in a first state according to an embodiment of the present invention.

FIG. 21B illustrates a perspective view of the tube-shaped reservoir having a closed end of FIG. 21A in a second state according to an embodiment of the present invention.

FIG. 21C illustrates a perspective view of a tube-shaped reservoir having a closed end of FIG. 21A in a third state according to an embodiment of the present invention.

FIG. 22 illustrates a perspective view of a reservoir having an internal spring according to an embodiment of the present invention.

FIG. 23 illustrates a perspective view of a reservoir having an internal cage according to an embodiment of the present invention.

FIG. 24 illustrates a perspective view of a reservoir having internal rings according to an embodiment of the present invention.

FIG. 25 illustrates a perspective view of a reservoir having an external shell according to an embodiment of the present invention.

FIG. 25A illustrates a cross-sectional view of the reservoir having the external shell of FIG. 25 according to an embodiment of the present invention.

FIG. 26 illustrates a cross-sectional view of another reservoir having an external shell according to an embodiment of the present invention.

FIG. 27 illustrates a perspective, cross-sectional view of a reservoir having depressions according to an embodiment of the present invention.

FIG. 28 illustrates a cross-sectional view of a reservoir having depressions according to an embodiment of the present invention.

FIG. 29 illustrates a cross-sectional view of a reservoir having depressions according to an embodiment of the present invention.

FIG. 30A illustrates a top view of a gastric banding system having a compliant reservoir about the circumference of the gastric band according to an embodiment of the present invention.

FIG. 30B illustrates a side view of the gastric banding system of FIG. 30A according to an embodiment of the present invention.

FIG. 31 illustrates a side view of a gastric banding system of having a compliant reservoir about the circumference of the gastric band according to an embodiment of the present invention.

FIG. 32 illustrates an orientation of a gastric banding system having a plurality of reservoirs extending radially from the gastric band according to an embodiment of the present invention.

FIG. 33A illustrates a large bolus/small bolus pressure-time graph for two distinct gastric banding systems.

FIG. 33B illustrates a large bolus/small bolus pressure-time graph for two distinct gastric banding systems.

FIG. 34A illustrates a perspective view of a self-contained gastric banding system according to an embodiment of the present invention.

FIG. 34B illustrates a top view of the self-contained gastric banding system of FIG. 34A according to an embodiment of the present invention.

FIG. 35 illustrates a top perspective view of another self-contained gastric banding system according to an embodiment of the present invention.

FIG. 36 illustrates an exploded, perspective view of a gastric banding system having gas-impermeable components according to an embodiment of the present invention.

FIG. 37A illustrates an exploded, perspective view of a hybrid gas-fluid gastric banding system having gas-impermeable components according to an embodiment of the present invention.

FIG. 37B illustrates an exploded, perspective view of a hybrid foam-fluid gastric banding system having gas-impermeable components according to an embodiment of the present invention.

FIG. 38A illustrates an exploded, perspective view of a hybrid gas-fluid gastric banding system having a gas-spring according to an embodiment of the present invention.

FIG. 38B illustrates a close-up view of the gas-spring within a reservoir of FIG. 38A according to an embodiment of the present invention.

FIG. 38C illustrates a close-up view of the gas-spring of FIG. 38A according to an embodiment of the present invention.

FIG. 38D illustrates a cross-sectional view of the gas-spring of FIG. 38A according to an embodiment of the present invention.

FIG. 39 illustrates a close up view of an access port for a gastric banding system according to an embodiment of the present invention.

FIG. 40 illustrates a close up view of another access port for a gastric banding system according to an embodiment of the present invention.

FIG. 41 illustrates a close up view of another access port for a gastric banding system according to an embodiment of the present invention.

FIG. 42 illustrates a perspective view of a gastric banding system having a flow rate restrictor according to an embodiment of the present invention.

FIG. 43 illustrates a time-pressure curve for a gastric banding system without a flow rate restrictor and a time-pressure curve for a gastric banding system with a flow rate restrictor.

FIG. 44A illustrates an always-open gastric banding system according to an embodiment of the present invention.

FIG. 44B illustrates an always-open gastric banding system having an elastic member according to an embodiment of the present invention.

FIG. 45 illustrates a cross-sectional view of a gastric band located about an esophageal-gastric junction of a patient.

FIG. 46 illustrates a cross-sectional view of a gastric band having a funnel shape according to an embodiment of the present invention.

FIG. 47A illustrates an exploded, perspective view of a gastric banding system including a gastric band without a ring according an embodiment of the present invention.

FIG. 47B illustrates a perspective view of the gastric band of FIG. 47A according to an embodiment of the present invention.

FIG. 48A illustrates an exploded, perspective view of a gastric banding system including a gastric band having a ring with holes according an embodiment of the present invention.

FIG. 48B illustrates a perspective view of the gastric band of FIG. 48A according to an embodiment of the present invention.

FIG. 49A illustrates an exploded, perspective view of a gastric banding system including a gastric band having a ring with cut-out portions according an embodiment of the present invention.

FIG. 49B illustrates a perspective view of the gastric band of FIG. 49A according to an embodiment of the present invention.

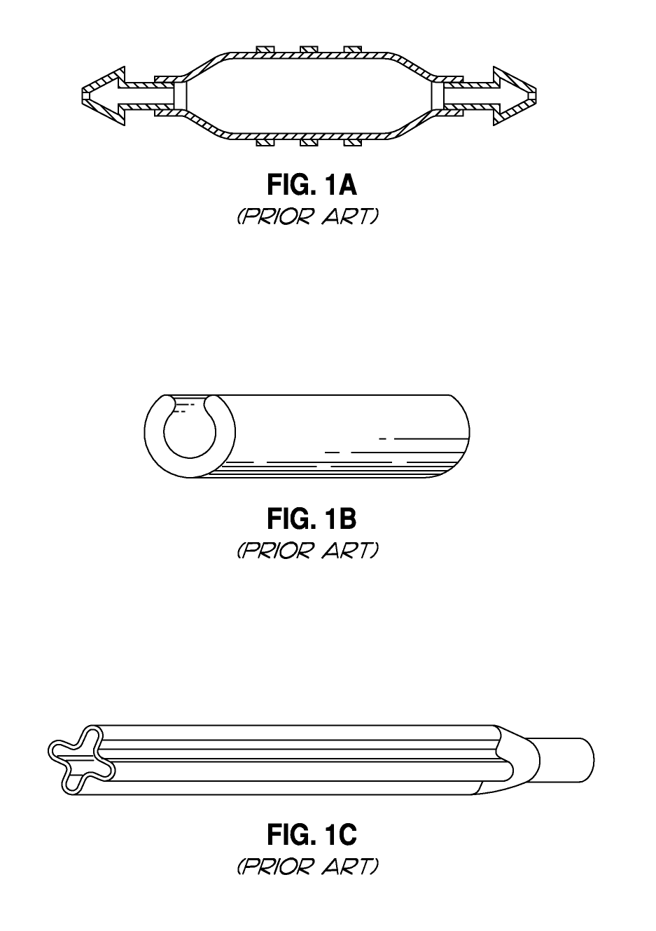

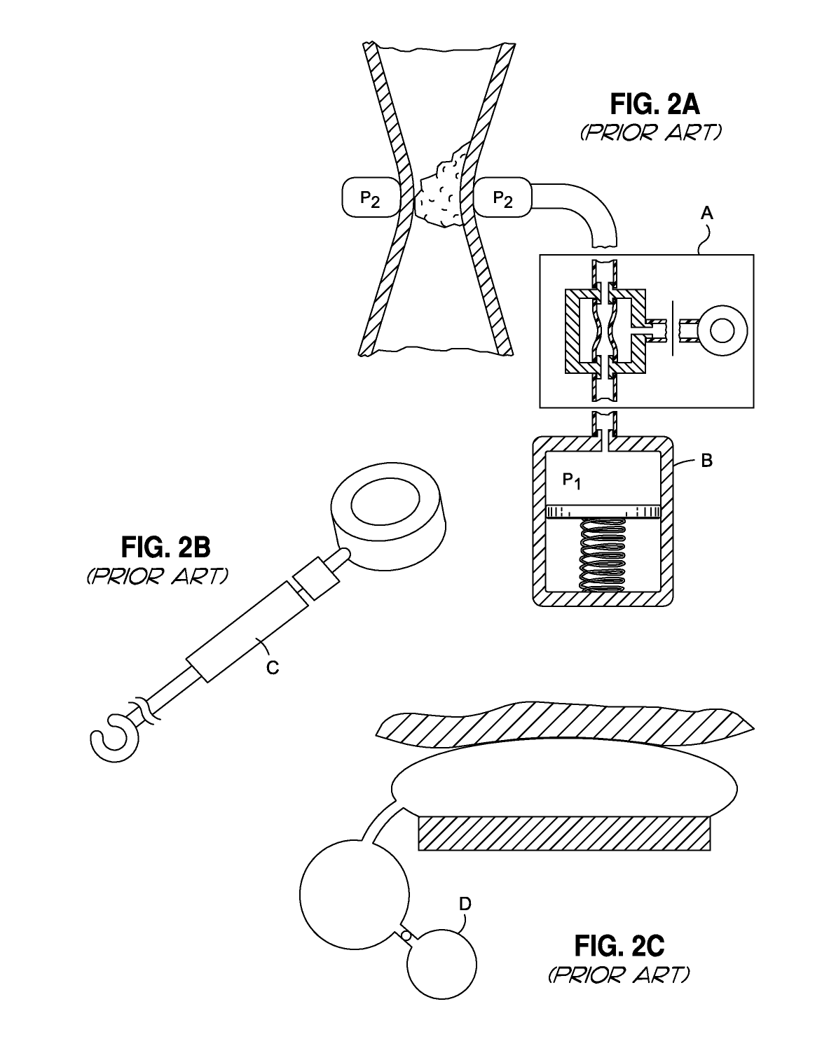

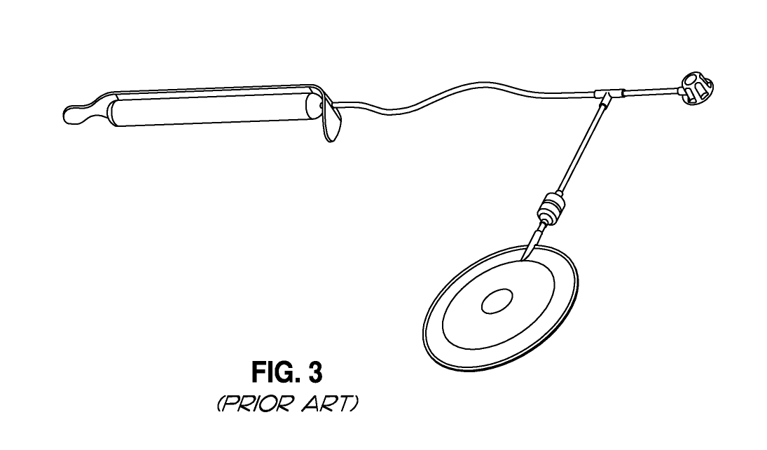

### DETAILED DESCRIPTION

Apparatuses, systems and/or methods that implement the embodiments of the various features of the present invention will now be described with reference to the drawings. The drawings and the associated descriptions are provided to illustrate some embodiments of the present invention and not to limit the scope of the present invention. Throughout the drawings, reference numbers are re-used to indicate correspondence between referenced elements.

The present invention generally provides self-adjusting gastric banding systems, for example, for treatment of obesity and obesity related conditions, as well as systems for allowing automatic self-adjustment of gastric bands when a patient swallows a large bolus.

Self-adjusting gastric bands are effective in helping a patient lose weight when the band is properly tightened around the patient's esophageal-gastric junction. During normal operation, the band applies pressure to the outer surface of the upper stomach. But, in some instances, the patient may swallow a bolus which is too large to pass through the constriction produced by the band—for example, when the patient swallows a large piece of steak. The result can be a painful experience which, if it persists, may require medical intervention to release the blockage.

In accordance with various embodiments of the present invention, the self-adjusting gastric band provides the needed pressure to the stomach to encourage weight loss. However, when a large bolus of food is swallowed, the self-adjusting gastric band temporarily and automatically opens up to allow the bolus through. After the bolus passes through, the mechanisms within the band return the band to its original size and shape. In an embodiment, electrical power and/or power external to the patient is not utilized to perform these adjustments. Further, in an embodiment, complicated fluid control mechanisms, flow rate limiting devices, and/or valves are not utilized to regulate the transfer of fluid within the self-adjusting gastric band.

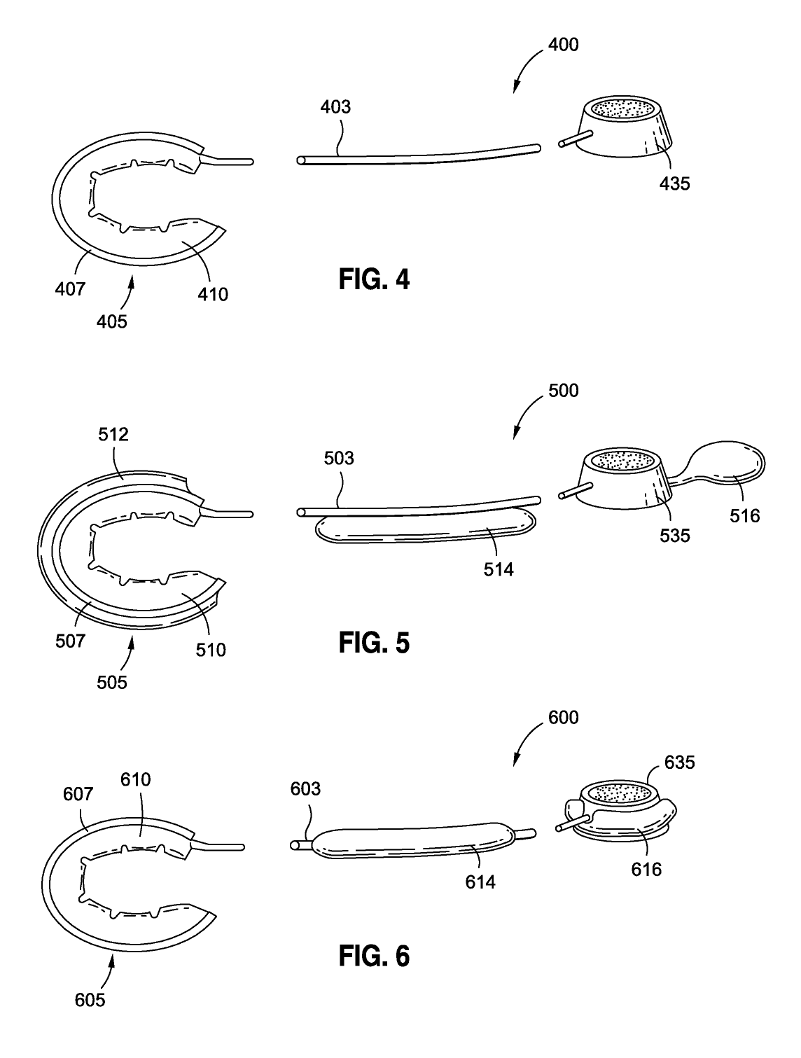

Turning now to FIG. 4, a self-adjusting gastric banding system 400 includes a gastric band 405 coupled to a subcutaneous injection port 435 via tubing 403. The gastric band 405 includes a circular ring 407 and an inflatable portion 410 disposed on the inside of the ring 407. The inflatable portion 410 separates the patient's stomach from the ring 407 when the gastric band 405 is implanted around the esophageal-gastric junction of the patient's stomach. The ring 407 provides structure and support to the inflatable portion 410, and facilitates implanting the gastric band 405 around the patient's stomach.

The access port 435 may be sutured onto the rectus muscle sheath or any other conveniently accessible muscle. The rectus muscle sheath provides a secure surface on which to attach the access port 435 under a layer of fat that separates the patient's skin from the muscle.

The inflatable portion 410 may be filled and drained with a fluid via the tubing 403. For example, the tubing 403 may be connected to the subcutaneous access port 435 for filling and draining the inflatable portion 410 via subcutaneous injections. The inflatable portion 410 may also be coupled to a reservoir to facilitate automatic adjustment of the inflatable portion 410, and the constriction it causes, when a large bolus attempts to pass through the constriction. When more fluid is introduced in the inflatable portion 410, the constriction around the stomach generally becomes tighter. Correspondingly, when less fluid is present, the constriction loosens and/or opens up.

The fluids used within the gastric band 405 may include any fluid that is biocompatible and incompressible. The fluid has no adverse effect on the patient in the unlikely event that a leak emanates from the system. The fluid can simply be water or any biocompatible polymer oil such as castor oil. In an example embodiment, the fluid is saline, a drug, and/or combinations thereof.

In an embodiment, the ring 407 is designed to be a compliant portion of the gastric band 405. For example, the ring 407 may flex and/or expand in response to a bolus of food moving through the constriction caused by the gastric band 405. The ring 407 may have flexible components and rigid components, such that the flexible components expand when a certain elevated and/or maximum pressure is reached in the inflatable portion 410. This elevated pressure may exist due to the presence of an obstruction such as a bolus near the gastric band 405. As the ring 407 expands, the diameters of the ring 407 and the inflatable portion 410 increase, and the constriction on the stomach due to the gastric band 405 is reduced to allow the bolus to pass through. When the bolus has passed, the elevated pressure no longer exists, and the gastric band 405 returns to the pre-obstruction state.

In another embodiment, the entire ring 407 may be flexible and/or expandable such that a diameter of the ring 407 increases in response to the elevated pressure in the inflatable portion 410. For example, the ring 407 may be constructed of silicone that has a durometer in the range of approximately 20 to approximately 70.

It should be understood that the flexible ring 407 and the other mechanisms disclosed herein for automatically adjusting the constriction of the gastric band 405 are only example embodiments. Any mechanism for automatically adjusting the constriction of the gastric band 405 that does not include electrical power, power external to the patient, complicated fluid control mechanisms, flow rate limiting devices, and/or valves is contemplated within the scope of the present invention. As an example, the term “automatically” refers to situations when the compliant member expands, moves, contracts or is altered without the use of an electronic device causing the change.

With reference to FIG. 5, various compliant components may be utilized to automatically adjust the constriction of the gastric band 505 about the esophageal-gastric junction of the patient's stomach. Although three compliant components are illustrated in FIG. 5, as noted above, one or more of the components may be present in various embodiments of the present invention.

For example, in an embodiment, a band compliant component 512 is fluidly coupled to the inflatable portion 510 of the gastric band 505. The compliant component 512 is located on the outside of the ring 507, opposite the inflatable portion 510, and may be coupled to the ring 507 and the inflatable portion 510. Further, in an embodiment, one or more fluid ports may extend from the inflatable portion 510 to the compliant component 512 to fluidly couple the inflatable portion 510 to the compliant component 512.

With reference to FIGS. 5 and 6, and in accordance with various embodiments, a tube compliant component 514, 614 may be fluidly coupled to the tubing 503, 603. As illustrated in FIG. 6, the compliant component 614 may run along substantially the entire length of the tubing 603. In another embodiment, as illustrated in FIG. 5, the compliant component 514 may be limited to a smaller section of the entire length of the tubing 503. The compliant component 514, 614 may be fluidly coupled to the tubing 503 at one or more locations. For example, with reference to FIG. 6, the compliant component 614 and the tubing 603 may be perforated to allow for efficient transfer of the fluid between the tubing 603 and the compliant component 614.

In another embodiment, the tubing 603 itself may be compliant, and the durometer, thickness, and/or diameter of the tubing 603 may be altered to achieve a desired degree of compliance. Other components of the gastric band 605 may similarly have altered properties in order to achieve a desired degree of compliance.

In an embodiment where the tube compliant component 514, 614 facilitates automated adjustment of the gastric band 505, 605, the compliant component 514, 614 may have features configured to resist kinking and/or leakage of the tubing 503, 603. For example, the compliant component 514, 614 may include rigid portions (e.g., similar to a skeleton) and flexible portions. The rigid components may give structure to the compliant component 514, 614 and/or the tubing 503, 603 to prevent kinking and/or leakage due to external forces on the compliant component 514, 614 and/or the tubing 503, 603. The flexible components may automatically expand in response to an increased pressure in the inflatable portion 510, 610 of the gastric band 505, 605.

In accordance with another embodiment, and with continued reference to FIGS. 5 and 6, the access port 535, 635 may be fluidly coupled to a port compliant component 516, 616. As illustrated in FIG. 5, the compliant component 516 may be a balloon, reservoir, or other expandable device that is adjacent to the port 535. In an embodiment as illustrated in FIG. 6, the compliant component 616 may substantially surround the access port 635. The compliant component 616 may be fluidly coupled to the access port 635 at a single location near a coupling between the tubing 603 and the access port 635. In another embodiment, the compliant component 616 may be fluidly coupled to the access port 635 at multiple locations.

As noted above, any combination of the inflatable portion compliant component 512, a compliant ring 407, the tube compliant component 514, 614, and/or the port compliant component 516, 616 may be used in accordance with various embodiments. When the pressure in the inflatable portion 510, 610 exceeds a predetermined pressure, the compliant components 407, 512, 514, 516, 614, 616, in any particular configuration or combination, expand to receive an amount of the fluid from the inflatable portion 510, 610 via the inflatable portion 510, 610, the tubing 503, 603, and/or the access port 535, 635, and/or to reduce the constriction formed by the gastric band 405, 505, 605. The predetermined pressure may be predetermined based on a pressure that would indicate an obstruction is attempting to pass through the constriction caused by the gastric band 405, 505, 605.

The compliant components 407, 512, 514, 516, 614, 616 described herein, in accordance with various embodiments, may be designed with an expansion pressure at which pressure the components 407, 512, 514, 516, 614, 616 begin to expand, to receive fluid from the inflatable portion 510, 610 of the gastric band 505, 605, and/or to reduce the constriction formed by the gastric band 405, 505, 605. The expansion pressure may be configured to correspond to a predetermined pressure in the inflatable portion 410, 510, 610 that may indicate an obstruction exists in the esophageal-gastric junction.

For example, the obstruction may result in a large spike in intra-esophageal pressure that exceeds the expansion pressure and causes the compliant components to expand and receive fluid from the inflatable portion 510, 610. The reduction in fluid in the inflatable portion 510, 610 causes the constriction around the patient's stomach to loosen, in order to relieve the spike in pressure and allow the obstruction to pass through the esophageal-gastric junction. When the obstruction passes, the increased pressure in the inflatable portion 510, 610 is reduced, and the fluid flows back into the inflatable portion 510, 610 due to the elasticity of the compliant components 512, 514, 516, 614, 616, to restore the original amount of constriction of the gastric band 505, 605. This change in constriction of the gastric band 505, 605 results or is achieved without the use of flow rate limiting devices or valves.

The various compliant components disclosed herein may have any shape or configuration that facilitates removing an amount of fluid from the inflatable portion of the gastric band in response to an increased pressure in the inflatable portion. For example, the compliant components may be selected from a group consisting of a compressible reservoir, an elastic polymer, a balloon, a rubber container, a silicone container, a collapsible container, a bellows, and combinations thereof that are configured to contain the fluid.

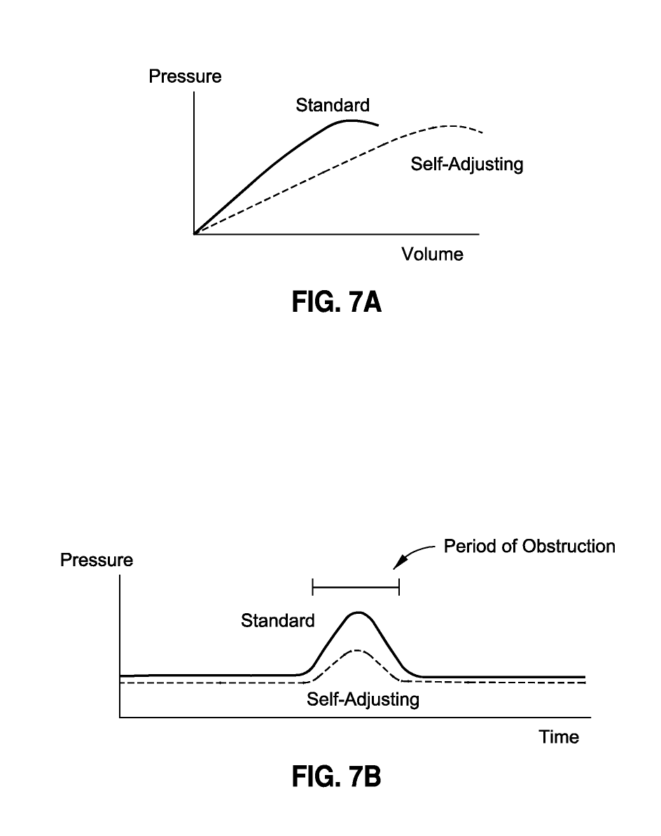

The graph in FIG. 7A illustrates, according to various embodiments, the effect the compliant components described herein have on the pressure in the gastric banding system. As can be seen in FIG. 7A, a standard gastric banding system without compliant components has a certain pressure-volume relationship. After the gastric banding system is flushed with saline to remove any air trapped within the system (e.g., in the gastric band, the tubing, and the port), the pressure-volume relationship generally takes the form illustrated by the “Standard” curve in FIG. 7A. The dashed “Compliant” curve illustrates an example embodiment of the pressure-volume relationship for a gastric banding system with one or more compliant components. As illustrated, the self-adjusting gastric banding system may include a greater volume of saline than a standard gastric banding system for a given level of pressure.

The graph in FIG. 7B illustrates, according to various embodiments, pressure characteristics of a “Standard” gastric banding system and a “Self-Adjusting” gastric banding system during use of the systems in a patient. Initially, the two systems are set to the same operating pressure, for example, for a desired level of constriction of the patient's stomach. As a large bolus of food or some other obstruction encounters the gastric band, the pressure in each system increases. As illustrated, the standard system has a larger pressure increase during the period of obstruction than the self-adjusting gastric banding system experiences. This smaller increase in pressure, according to various embodiments, is due to the addition of the reservoir space in the compliant component(s). As pressure in the gastric banding system increases, fluid is transferred into the reservoir space. Once the obstruction passes, the fluid is automatically returned from the reservoir space back into the gastric band.

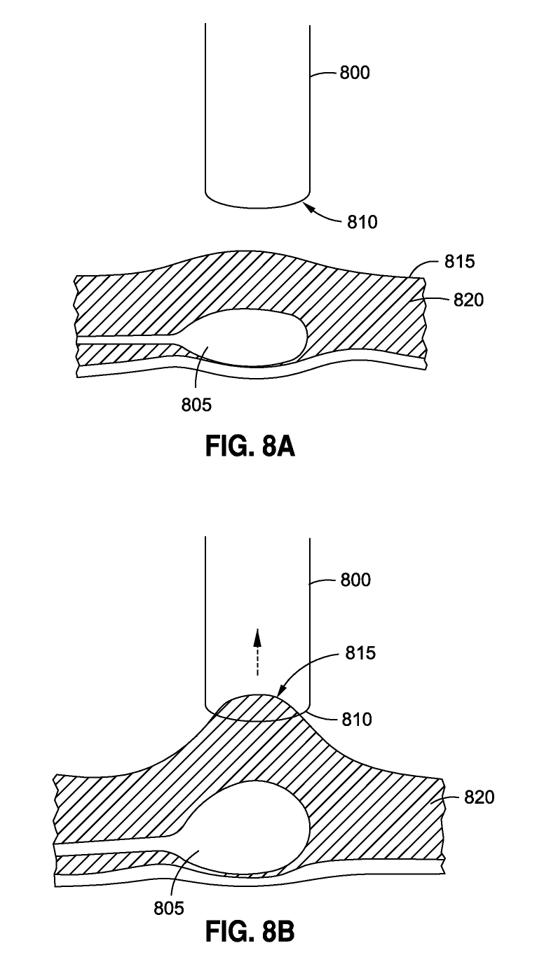

FIG. 8A illustrates an embodiment of a vacuum device 800 which may be used to assist the patient in transferring fluid into a reservoir 805 from a gastric band (not shown) to allow a large bolus to pass through the constriction of the gastric band. The vacuum device 800 may include a tip 810, which in one embodiment, may have a diameter substantially equal to the diameter of the reservoir 805.

As shown in FIG. 8B, when the vacuum device 800 is activated and brought close to a patient's skin 815, the vacuum device 800, by using suction, may form a seal on a contacted area of the patient's skin 815 and slightly pull or tug on the patient's skin 815 and a subcutaneous fat layer 820 located below the skin 815. The vacuum device 800 may be configured to provide suction strength of differing magnitudes, but without harming the patient. Since there is no pathway for air or bodily fluids to collect between the subcutaneous fat layer 820 of the patient and the top surface of the reservoir 805, the top surface of the reservoir 805 may be pulled towards the vacuum device 800 along with the subcutaneous fat layer 820. As a result of the suction created, fluid may flow out of the gastric band (not shown) and into the reservoir 805 thereby relaxing a constriction caused by the gastric band and allowing a bolus of food to pass through the constriction.

Specific embodiments of a compliant self-adjusting gastric banding system having been described, attention will now be turned to additional and/or alternative improvements which may be integrated and/or implemented with any number of obesity-preventing systems, including the self-adjusting gastric banding systems described herein.

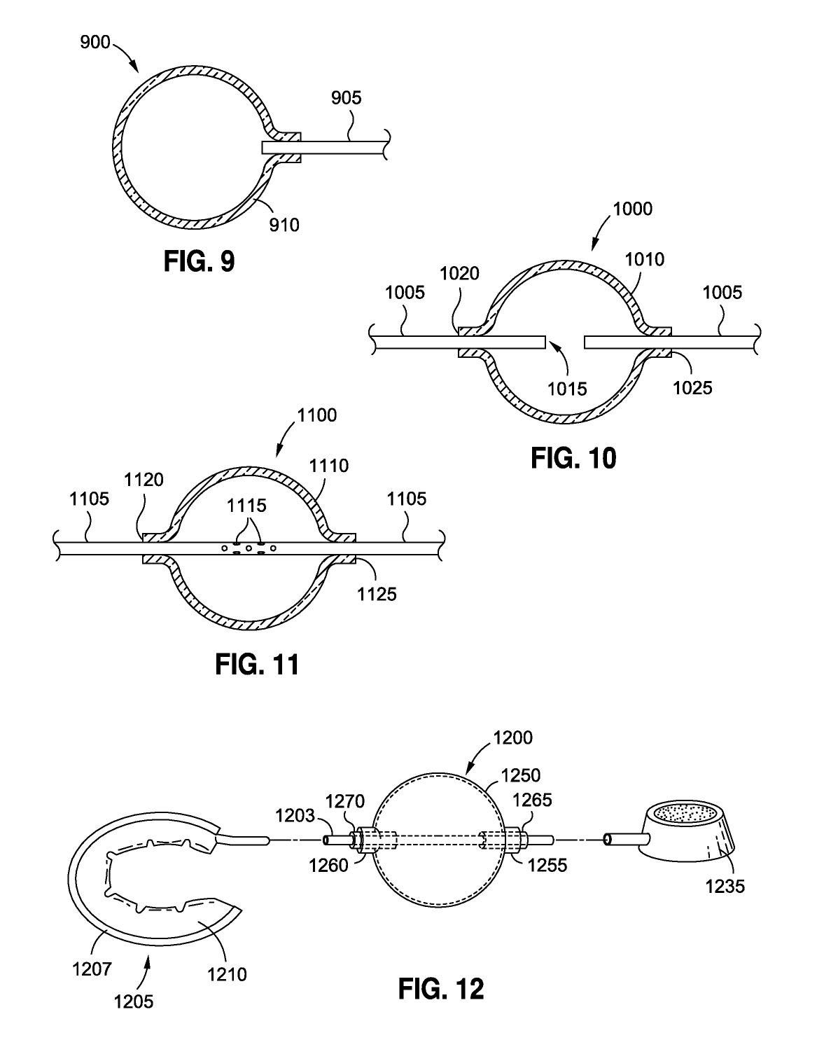

Turning to FIG. 9, a close-up view of a compliant tubing-reservoir system 900 is illustrated. The compliant tubing-reservoir system 900 may include a tubing 905 which terminates at or inside a balloon or a reservoir 910. In one embodiment, the tubing (not shown in FIG. 5) may connect an injection port (e.g., injection port 535 of FIG. 5) and a reservoir (e.g., port compliant portion 516 of FIG. 5) thereby allowing a fluid path to travel through the tubing 905 to the reservoir 910. While shown here in FIG. 9 to terminate in the reservoir 910, the tubing 905 may, in one embodiment, continue and pass through. In operation, the tubing-reservoir system 900 may relieve increases in pressure, for example, generated by the passing of a large bolus swallowed by the patient. The relief may result from the transfer of fluid or other substances from an inflatable portion (not shown) through the tubing 905 and into the elastically deformable balloon or reservoir 910.

FIG. 10 illustrates one embodiment of a fluid path connecting component for a pass-through tubing-reservoir system 1000 where a tubing 1005 passes through both a first opening 1020 and a second opening 1025 of a balloon or reservoir 1010. Here, the tubing 1005 may include a gap or cut 1015 inside the reservoir 1010 (which creates a fluid path between the tubing 1005 and the reservoir 1010). The gap 1015 is sealed within the reservoir 1010 such that the addition of fluid may enlarge the reservoir 1010 while the reduction of fluid may shrink or decrease the size of the reservoir 1010. In operation, the tubing-reservoir system 1000 may relieve increases in pressure, for example, generated by the passing of a large bolus swallowed by the patient. The relief may result from the transfer of fluid or other substances from an inflatable portion (not shown) through the tubing 1005 and into the elastically deformable reservoir 1010.

FIG. 11 illustrates an embodiment of a pass-through tubing-reservoir system 1100 where a tubing 1105 passes through both a first opening 1120 and a second opening 1125 of a balloon or reservoir 1110. The tubing 1105 is shown with a series of holes or slits 1115 located at a position inside the reservoir 1110 which allows fluid and/or other substances to exit the tubing 1105 and into the reservoir 1110. The same holes or slits 1115 further allow fluid and/or other substances to enter the tubing 1105 from the reservoir 1110, thereby creating the fluid path between the tubing 1105 and the reservoir 1110. In operation, the tubing-reservoir system 1100 may relieve increases in pressure, for example, generated by the passing of a large bolus swallowed by the patient. The relief may result from the transfer of fluid or other substances from an inflatable portion (not shown) through the tubing 1105 and into the elastically deformable balloon or reservoir 1110.

FIG. 12 illustrates an embodiment of a compliant pass-through tubing-reservoir system 1200 where a tubing 1203 passes through both a first opening 1255 and a second opening 1260 of a balloon or reservoir 1250. The system 1200 may also include a first collar 1265 and a second collar 1270 for facilitating the connection of the reservoir 1250 to the tubing 1203. The tubing 1203 may be fixed to the reservoir 1250 by the customizably-fitted first collar 1265 and the customizably-fitted second collar 1270. Here, the tubing 1203 and/or the reservoir 1250 may be compliant components creating a fluid path between a gastric band 1205 and a port 1235. In one embodiment, the gastric band 1205 and the port 1235 may be the gastric band 405 and the port 435 of FIG. 4, respectively. The gastric band 1205 may include a circular ring 1207 and an inflatable portion 1210. Similarly, the circular ring 1207 and the inflatable portion 1210 may be the circular ring 407 and the inflatable portion 410 of FIG. 4, respectively. In operation, the tubing-reservoir system 1200 may relieve increases in pressure, for example, generated by the passing of a large bolus swallowed by the patient. The relief may result from the transfer of fluid or other substances from the inflatable portion 1210 through the tubing 1203 and into the elastically deformable reservoir 1250.

In one embodiment, the balloons/reservoirs 910, 1010, 1110 and 1250 may be a spherical balloon constructed of biocompatible, elastomeric material. However, the balloon/reservoirs 910, 1010, 1110 and 1250 may be any other shape (e.g., a cylindrical balloon, etc.).

In one embodiment, each of the tubing-reservoir systems 900, 1000, 1100 and 1200 may be considered a compliant system having one or more compliant components. Moreover, the systems 900, 1000, 1100 and 1200 may be arranged with other components of a gastric banding system (e.g., gastric banding system 500 or 600) in series or in parallel with each other to optimize the overall compliance, and to add redundancy to the gastric banding system (e.g., gastric banding system 500 or 600), if desired.

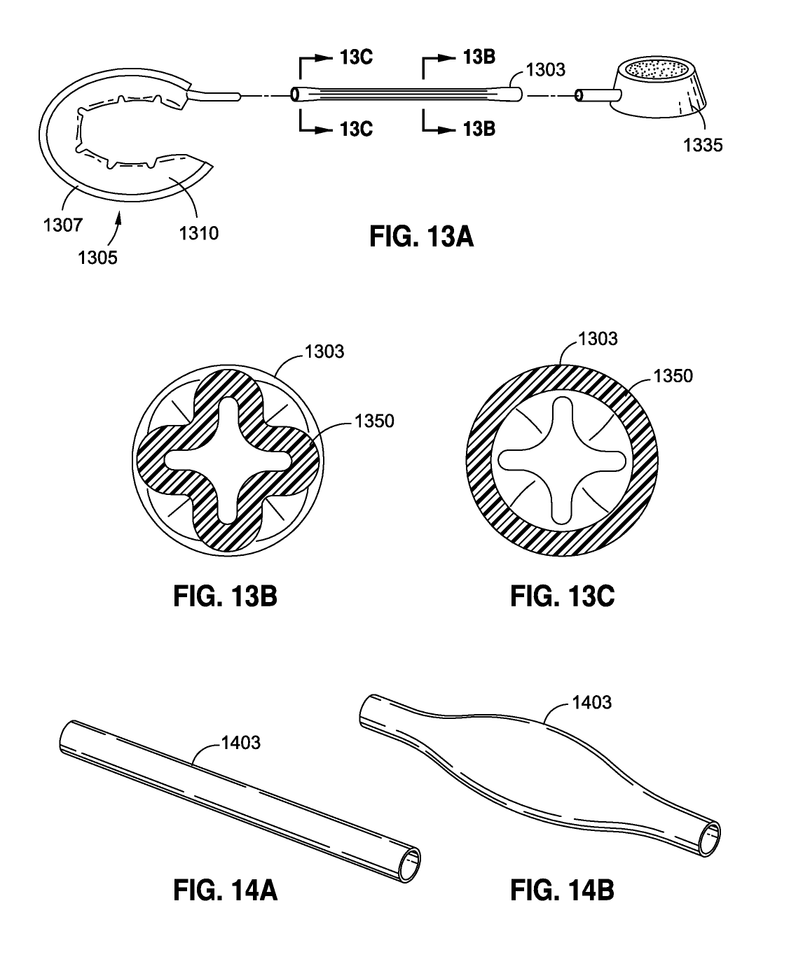

FIGS. 13A-C illustrate one embodiment of a compliant gastric banding system 1300 having a compliant reservoir 1303. The compliant gastric banding system 1300 may further include a gastric band 1305 having a ring 1307 and an inflatable portion 1310 fluidly coupled to an access port 1335 via the reservoir 1303. As shown, the reservoir 1303 may have a tube-like appearance and may replace a balloon-type reservoir (e.g., reservoir/balloon 910, 1010, 1110, 1250) as a compliant component in any gastric banding system (e.g., gastric banding systems 900, 1000, 1100, 1200). FIG. 13A generally illustrates the reservoir 1303 in an as-extruded view. The reservoir 1303, as further shown by FIGS. 13B and 13C, may include a star-shaped structure defining an outer circumference 1350 and may be sized to expand when holding fluid that is displaced from an adjacent non-compliant fluid-carrying device (e.g., the inflatable portion 1310). In operation, the reservoir 1303 may include star-shaped folds when in a natural, deflated state as shown in FIG. 13B. However, as fluid and/or other substances begin to move into the reservoir 1303 and exert pressure on the inner walls, the star-shaped folds may deform and expand, thereby changing the appearance of the outer circumference 1350 as shown in FIG. 13C. Here, the outer circumference 1350 may appear more similar to a normal, cylindrical tube. As the fluid moves out of the reservoir 1303, the star-shaped folds may begin to re-appear and the outer circumference 1350 may revert back to its star-shaped form as shown in FIG. 13B. The reservoir 1303 may be constructed from a polymer such as silicone, in various durometers so as to expand at a controlled rate.

FIGS. 14A and 14B illustrate one embodiment of a compliant tube-like reservoir 1403 in an as-molded state (e.g., when not overly filled with a fluid) and an expanded state (e.g., when filled with a fluid above a threshold volume), respectively. The reservoir 1403 may replace a balloon-type reservoir (e.g., reservoir/balloon 910, 1010, 1110, 1250) or another tube-like reservoir (e.g., reservoir 1303) as a compliant component for any gastric banding system (e.g., gastric banding systems 900, 1000, 1100, 1200, 1300). In operation, the reservoir 1403 may appear very similar to a non-compliant, stiff tube when in a natural, as-molded state as shown in FIG. 14A. However, as fluid and/or other substances begin to move into the reservoir 1403 and exert pressure on the inner walls, the elastic wall of the reservoir 1403 may begin to deform and expand, thereby appearing more similar to the expanded state as shown in FIG. 14B. As the fluid moves out of the reservoir 1403, the elastic walls may begin to regain its original, as-molded appearance. Similar to the reservoir 1303 of FIGS. 13A-C, the reservoir 1403 may be constructed from a polymer such as silicone, in various durometers so as to expand at a controlled rate. In one embodiment, the reservoir 1403 may have a uniform diameter of between about 1 and 100 millimeters in a natural, as-molded state and may inflate to a diameter of about 10 to 1000 millimeters in an expanded state (as measured at the location of the greatest diameter).

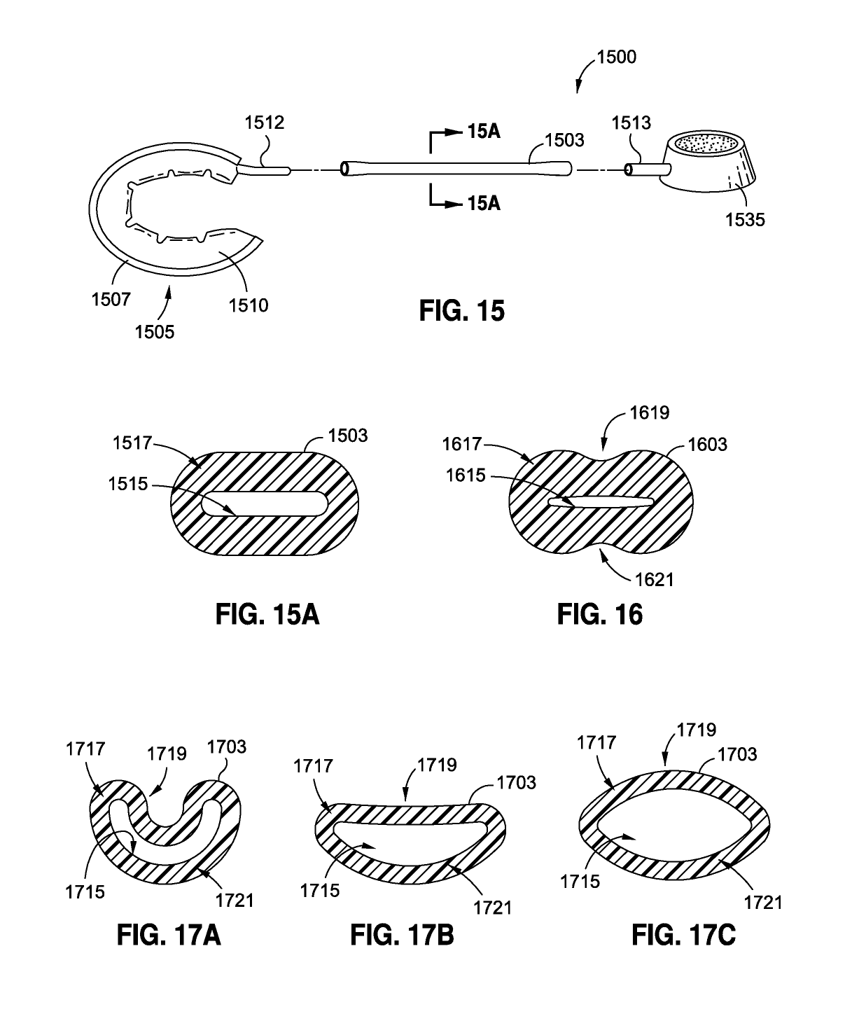

FIG. 15 illustrates one embodiment of a compliant gastric banding system 1500 having a compliant reservoir 1503. The compliant gastric banding system 1500 may further include a gastric band 1505 having a ring 1507 and an inflatable portion 1510 fluidly coupled to an access port 1535 via the reservoir 1503. As shown, the reservoir 1503 may have a flattened tube-like appearance and may replace a balloon-type reservoir (e.g., reservoir/balloon 910, 1010, 1110, 1250) as a compliant component in any gastric banding system (e.g., gastric banding systems 900, 1000, 1100, 1200). As shown, the reservoir 1503 may appear to have a flattened, tubular structure and may be sized to expand and hold fluid displaced from an adjacent non-compliant reservoir (e.g., inflatable portion 1510). The reservoir 1503 may be a fluid conduit for fluid transfer between the gastric band 1505 and the access port 1535. In one embodiment, the reservoir 1503 may be designed to be the compliant component and may function to allow for expansion so that unwanted fluid may move from a coupled stiffer tube (e.g., tube 1512 or 1513), thus reducing the amount of fluid in the stiffer tube (e.g., the tube 1512 or 1513), for a short period of time, when pressure is applied to the fluid within the stiffer tube (e.g., the tube 1512 or 1513).

FIG. 15A is a cross-sectional view of the reservoir 1503 of FIG. 15 and illustrates that a cross-sectional area 1515 within the reservoir 1503 where the fluid flows is continuous and smaller than a cross-sectional area 1517 of the reservoir 1503 itself. In addition to providing a unique, expandable shape profiled over a continuous length, improved performance may be achieved through reduction of the effects of the external forces on fluid within an adjacent, non-compliant component.

In one embodiment, the reservoir 1503 of FIG. 15 may be replaced by a reservoir 1603 of FIG. 16. A cross-section of the tube 1603 is illustrated in FIG. 16. As shown, a cross-sectional area 1615 within the reservoir 1603 where the fluid flows is continuous and smaller than a cross-sectional area 1617 of the tube reservoir itself. In addition to providing a unique, expandable shape profiled over a continuous length, improved performance may be achieved through reduction of the effects of the external forces on fluid within an adjacent, non-compliant component. As compared to the reservoir 1503 of FIG. 15, the tube 1603 may, in one embodiment, have a smaller cross-sectional area 1617 where fluid may flow such that, an increase in fluid may cause indented portions 1619 and 1621 to expand.

In one embodiment, the reservoir 1503 of FIG. 15 may be replaced with a reservoir 1703 of FIG. 17A. A cross-section of the reservoir 1703 is illustrated in FIG. 17A. As shown, a cross-sectional area 1715 within the reservoir 1703 where the fluid flows is continuous and smaller than a cross-sectional area 1717 of the reservoir 1703 itself. In addition to providing a unique, expandable shape profiled over a continuous length, improved performance may be achieved through reduction of the effects of the external forces on fluid within an adjacent, non-compliant component. As compared to the reservoir 1503 of FIG. 15, the reservoir 1703 may, in one embodiment, include a U-shaped tubular structure sized to expand such that a top portion 1719 may be configured to expand from a U-shaped curve to a flatter curve (as shown in FIG. 17B) when more fluid is added to the reservoir 1703. When even more fluid is added to the reservoir 1703 (e.g., beyond a certain volume threshold), the flatter curve may protrude outward (as shown in FIG. 17C). In one embodiment, as the top portion 1719 changes from the U-shaped curve to the flatter curve to the protruded curve as fluid is added, the bottom portion 1721 may expand longitudinally (e.g., straighten out) as shown in FIGS. 17B and 17C.

The reservoirs 1503, 1603 and 1703 may each be constructed out of a polymer such as silicon, in various durometers. By controlling the durometer of the constructed material, the expansion rates as fluid is added to the reservoirs 1503, 1603 and 1703 may be controlled.

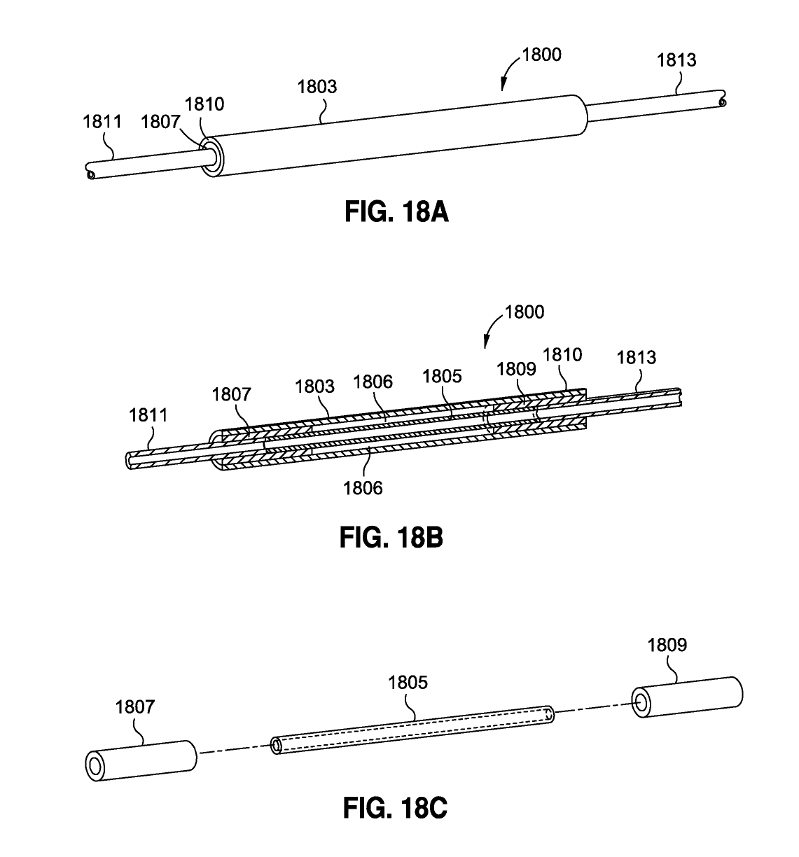

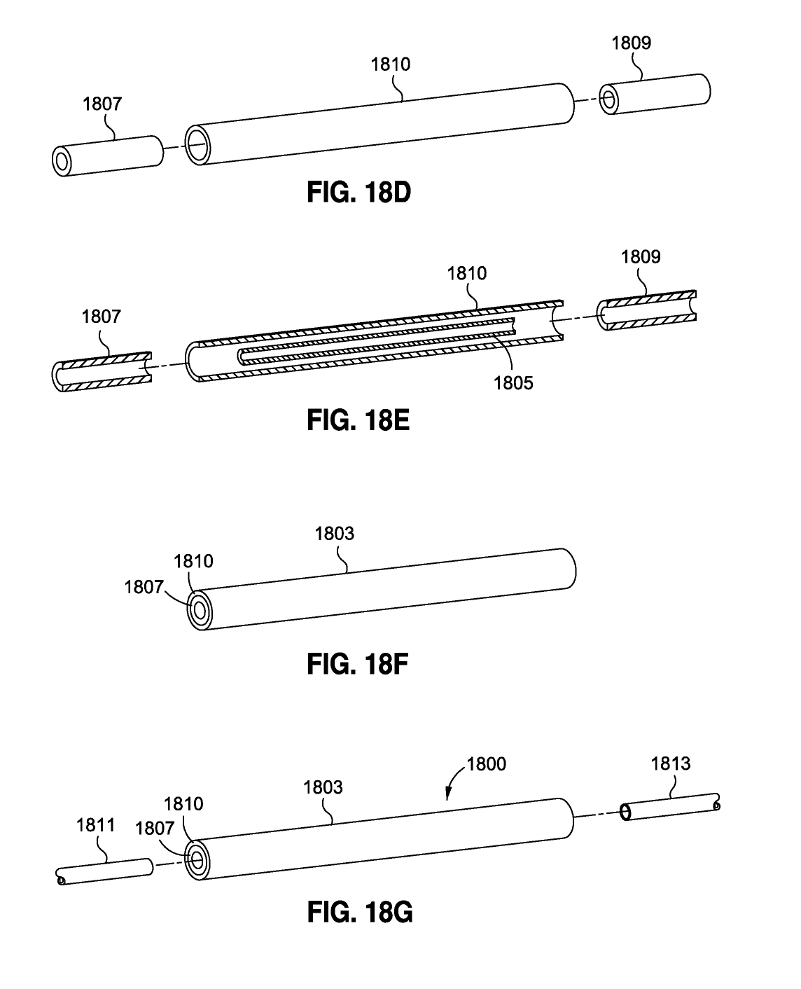

FIG. 18A illustrates an embodiment of a compliant tube-on-tube reservoir system 1800. The reservoir system 1800 may replace a balloon-type reservoir (e.g., reservoir/balloon 910, 1010, 1110, 1250) or another tube-like reservoir (e.g., reservoir 1303) as a compliant component for any gastric banding system (e.g., gastric banding systems 900, 1000, 1100, 1200, 1300). In one embodiment, the compliant tube-on-tube reservoir system 1800 may include a tube 1803 having an outer tube 1810 and joining portions (first joining portion 1807 shown here) configured to couple the tube 1803 (including an inner compliant tube 1805, shown in FIG. 18B) with non-compliant tube portions 1811 and 1813. FIG. 18B illustrates a cross sectional view of the system 1800, including the internal structure not shown in FIG. 18A. The inner compliant tube 1805 may enlarge when fluid is introduced into the cavity of the inner compliant tube 1805. The inner compliant tube 1805 may include a diameter substantially equivalent to the diameters of the non-compliant tube portions 1811 and 1813. The inner compliant tube 1805 may be coupled to the non-compliant tube portions 1811 and 1813 by the first joining portion 1807 and a second joining portion 1809. As shown, the joining portions 1807 and 1809 may be configured to have an inner circumference sized to fit both the inner compliant tube 1805 and the non-compliant tube portions 1811 and 1813. In one embodiment, the joining portions 1807 and 1809 do not contact one another, but instead form a gap 1806 between them. The gap 1806 may further be defined by the outer surface of the inner compliant tube 1805 and the inner surface of the outer tube 1810. The gap 1806 may serve as a sealed space for expansion of the inner compliant tube 1805 and to provide a buffer when the outer tube 1810 is compressed. The outer tube 1810 may, in one embodiment, be rigid, and may be further sized to fit the joining portions 1807 and 1809 within its internal cavity. Generally, as a patient's body presses on the non-compliant tube portions 1811 and 1813, and/or on the outer tube 1810, kinks, bending, or compression which disrupts fluid flow may be prevented.

FIGS. 18C-18G illustrate a method of constructing the system 1800 of FIGS. 18A and 18B. As shown in FIG. 18C, the inner compliant tube 1805 may be inserted into the first joining portion 1807 and the second joining portion 1809. In one embodiment, an adhesive may be used to attach the surfaces. However, other attachment methods may be practiced without the use of any adhesive (e.g., where the inner compliant tube 1805 is sized to fit tightly into the cavity of the joining portions 1807 and 1809). FIG. 18D illustrates the outer tube 1810 overlaying the joining portions 1807 and 1809. FIG. 18E illustrates a deconstructed view of the placement of the inner compliant tube 1805 with respect to the outer tube 1810. FIG. 18F illustrates the compliant tube 1803 as fully constructed. FIG. 18G illustrates the insertion of the two non-compliant tube portions 1811 and 1813 into the compliant tube 1803 to complete the tube-on-tube reservoir system 1800.

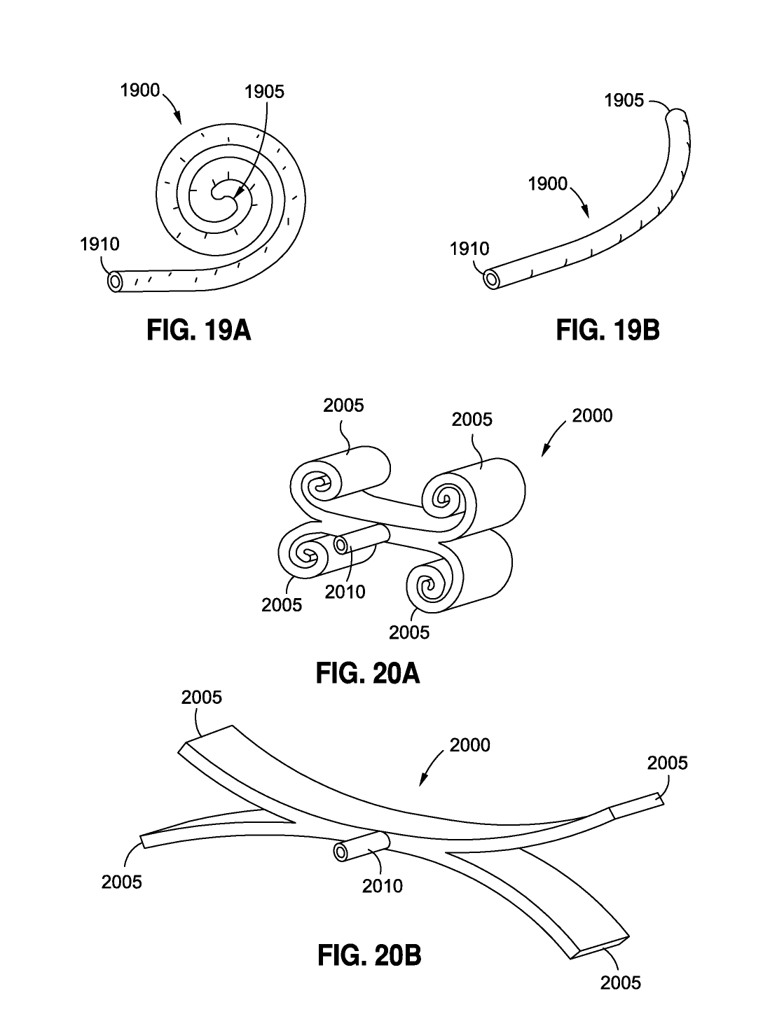

As discussed above with respect to FIGS. 5 and 6, port compliant components 516 and 616 may be used in accordance with various embodiments, such that when the pressure in the inflatable portion 510, 610 exceeds a predetermined pressure, the port compliant component 516 and 616 may expand to receive an amount of fluid from the inflatable portion 510, 610, the tubing 503, 603, and/or the access port 535, 635, and/or to reduce the constriction formed by the gastric band 505, 605. FIGS. 19A-19B, 20A-20B and 21A-21C illustrate additional compliant reservoir systems 1900, 2000 and 2100, respectively, that may be implemented as port compliant components 516 and 616.

The compliant reservoir systems 1900, 2000 and 2100 may be constructed such that a portion of their compliance comes from a conformational change or a shape-change in the systems 1900, 2000 and 2100. For example, as the pressure inside the reservoir systems 1900, 2000 and 2100 increases, one or more portions may move to a different location or change to a different state. More particularly, the systems 1900, 2000 and 2100 take on a different shape in a pressurized state.

With respect to FIG. 19A, the compliant reservoir system 1900 may be “coiled” in a precurved shape as shown in an unpressurized state. As the pressure increases inside the system 1900 (e.g., as fluid flows into the system 1900 through a distal end 1910), a proximal end 1905 of the coiled, precurved shape may begin to “uncoil” and straighten out as the system 1900 expands from a flat tube to a more rounded tube, as shown in FIG. 19B. As the pressure is removed, the system 1900 may reform and revert into the coil-like shape due to stresses in the material.

FIG. 20A illustrates a compliant reservoir system 2000 having a distal end 2010 and “coiled wings” 2005. As shown, system 2000 may have four “coiled wings” but any number of “coiled wings” may be implemented. The “coiled wings” in FIG. 20A are shown in an unpressurized state. As the pressure increases inside the system 2000 (e.g., as fluid flows into the system 2000 through a distal end 2010), each of the “coiled wings” 2005 may begin to uncoil and straighten out as the system 2000 expands when fluid is increasingly added causing the pressure. FIG. 20B illustrates the system 2000 in a pressurized state. Here, the “coiled wings” are straightened out and no longer in the coiled position. As pressure is removed, the system 2000 may reform and revert into the coil-like shape due to stresses in the material.

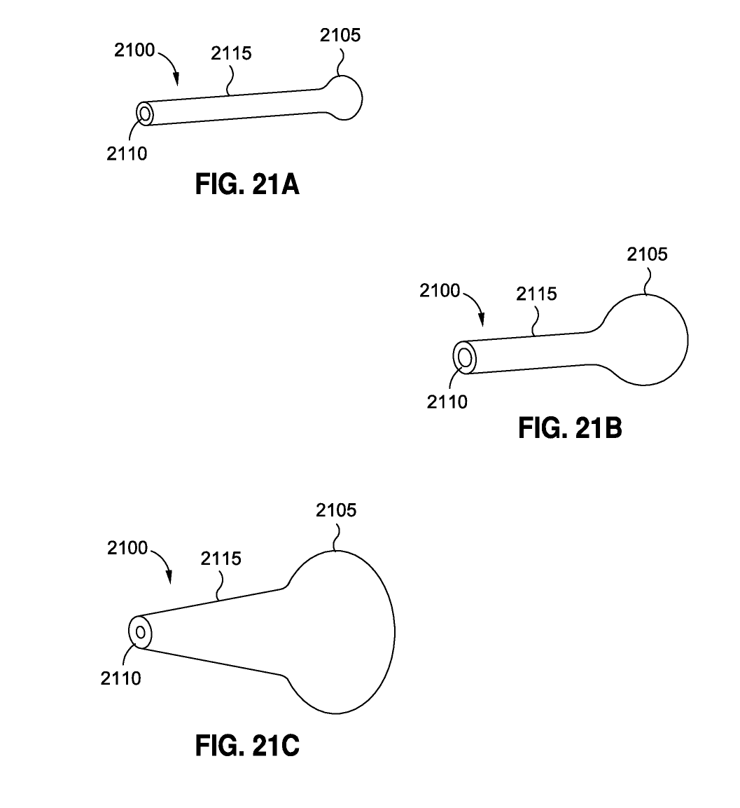

FIG. 21A illustrates a compliant reservoir system 2100 having a distal opening 2110, a body portion 2115 and a bulged proximal end 2105. As the pressure increases inside the system 2100 (e.g., as fluid flows into the system 2100 through the distal end 2110), the bulged proximal end 2105 may enlarge. FIG. 21B illustrates the system 2100 in a pressurized state with an enlarged bulged proximal end 2105 storing the fluid. Here, as the fluid collects in the bulged proximal end 2105, the bulged proximal end 2105 may further increase in size. In one embodiment, the body portion 2115 may remain substantially the same size or increase slightly in response to the introduction of fluid. FIG. 21C illustrates the system 2100 when a large amount of fluid is introduced into the system 2100, thereby causing the preferential inflation of the bulged proximal end 2105. However, as shown in FIG. 21C, other portions of the system 2100 may also incrementally increase in size such as the body portion 2115. As pressure is removed, the system 2100 may revert to the shape of FIG. 21A due to stresses in the material.

In addition to, or as an alternative to reservoirs with different shapes and structures (e.g., compliant reservoir system 1900, 2000 and 2100), embodiments of compliant reservoirs may include internal structures such as springs, cages and/or rings.

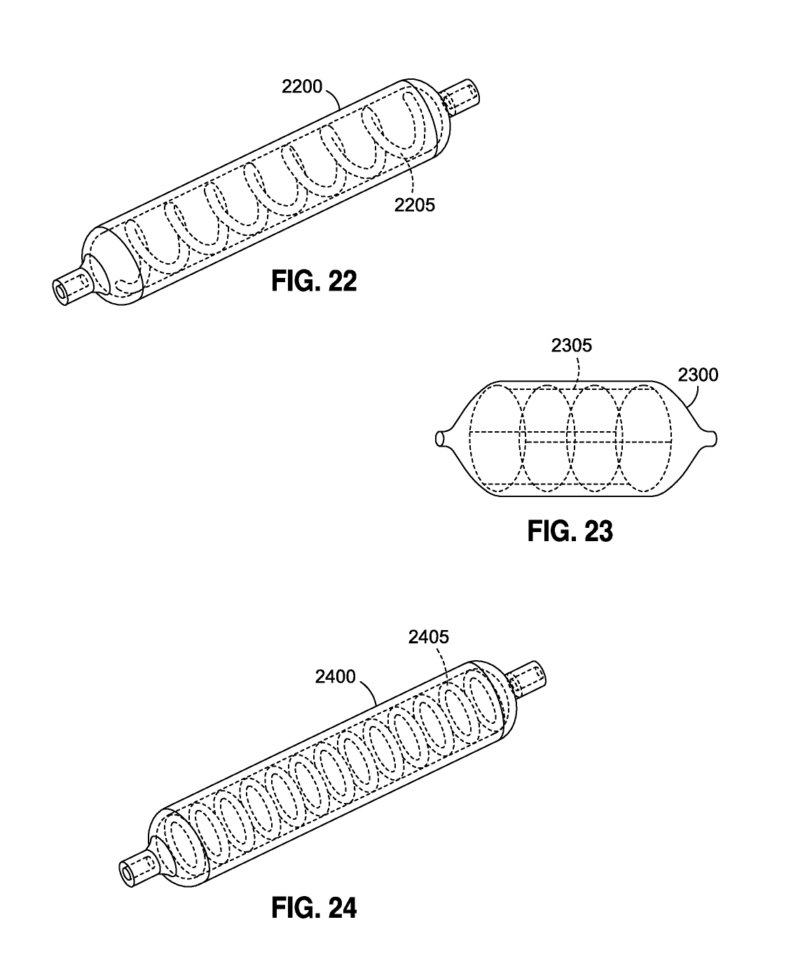

FIG. 22 illustrates an embodiment of a compliant reservoir 2200 which may be integrated into the tubing (e.g., tubing 403 or 503), added as an additional compliant component and/or attached to the access port (e.g., compliant component 516). As shown in FIG. 22, the compliant reservoir 2200 may include a spring 2205 which provides a skeletal structure. As the patient's body presses on the compliant reservoir 2200, the spring 2205 prevents the compliant reservoir 2200 from being kinked, bent or compressed in a way that disrupts flow of fluid or causes unwanted fluid to move into the inflatable portions (e.g., inflatable portions 510 and 610). In one embodiment, the spring 2205 may provide the compliant reservoir 2200 properties including high radial stiffness and low axial stiffness. The radial stiffness allows the compliant reservoir 2200 to support weight applied externally while the low axial stiffness provides for volumetric expansion of the compliant reservoir 2200 internally. In addition, the spring 2205 may provide internal support to resist loading from body tissues without an increase in pressure inside the compliant reservoir 2200. In one embodiment, the spring 2205 and the compliant reservoir 2200 may be configured to increase pressure relative to the volume displaced.

FIG. 23 illustrates one embodiment of a compliant reservoir 2300 having an internal structure in the form of a cage 2305. The compliant reservoir 2300 may be integrated into a tubing (e.g., tubing 403 or 503), added as an additional compliant component and/or attached to the access port (e.g., compliant component 516). As shown in FIG. 23, the compliant reservoir 2300 may include the internal cage 2305, which provides the compliant reservoir with a skeletal structure. As the patient's body presses on the compliant reservoir 2300, the rings and bars of the cage 2305 may prevent the compliant reservoir 2300 from being kinked, bent or compressed in a way that disrupts the flow of fluid or causes unwanted fluid to move into the inflatable portions (e.g., inflatable portions 510 and 610). In one embodiment, the cage 2305 may provide the compliant reservoir 2300 high radial stiffness and low axial stiffness. The radial stiffness may allow the compliant reservoir 2300 to support weight applied externally while the low axial stiffness provides for volumetric expansion of the compliant reservoir 2300 internally. In addition, the cage 2305 may provide internal support to resist loading from body tissues without an increase in pressure inside the compliant reservoir 2300. In one embodiment, the cage 2305 and the compliant reservoir 2300 may be configured to increase pressure relative to the volume displaced.

FIG. 24 illustrates one embodiment of a compliant reservoir 2400 having an internal structure in the form of parallel rings 2405. The compliant reservoir 2400 may be integrated into a tubing (e.g., tubing 403 or 503), added as an additional compliant component and/or attached to the access port (e.g., compliant component 516). As shown in FIG. 24, the compliant reservoir 2400 may include the parallel rings 2405, which provides the compliant reservoir 2400 with a skeletal structure. In one example, the parallel rings 2405 may be joined together. As the patient's body presses on the compliant reservoir 2400, the rings 2405 may prevent the compliant reservoir 2400 from being kinked, bent or compressed in a way that disrupts flow of fluid or causes unwanted fluid to move into the inflatable portions (e.g., inflatable portions 510 and 610). In one embodiment, the rings 2405 may provide the compliant reservoir 2400 high radial stiffness and low axial stiffness. The radial stiffness may allow the compliant reservoir 2400 to support weight applied externally while the low axial stiffness provides for volumetric expansion of the compliant reservoir 2400 internally. In addition, the rings 2405 may provide internal support to resist loading from body tissues without an increase in pressure inside the compliant reservoir 2400. In one embodiment, the rings 2405 and the compliant reservoir 2400 may be configured to increase pressure relative to the volume displaced.

While the internal structures (e.g., the spring 2205, the cage 2305 and the parallel rings 2405) of FIGS. 22, 23 and 24 may have different configurations, they may provide for different or same pressures. In one embodiment, the spring 2205, the cage 2305 and the parallel rings 2405 may be made of metals or polymers such as stainless steel, titanium, nitinol, PEEK, ultem, delrin, polycarbonate, polysulfone, among other materials. Materials used may further be combined to provide the desired properties. With respect to construction, the spring 2205, the cage 2305 and the parallel rings 2405 may be over-molded and/or contiguous with the compliant reservoir (e.g., compliant reservoirs 2200, 2300 and 2400).

In addition, or as an alternative to the internal structures of FIGS. 22-24, external structures may be utilized to protect a compliant reservoir from being kinked, bent or compressed in a way that disrupts flow of fluid or causes unwanted fluid to move into the inflatable portions (e.g., inflatable portions 510 and 610).

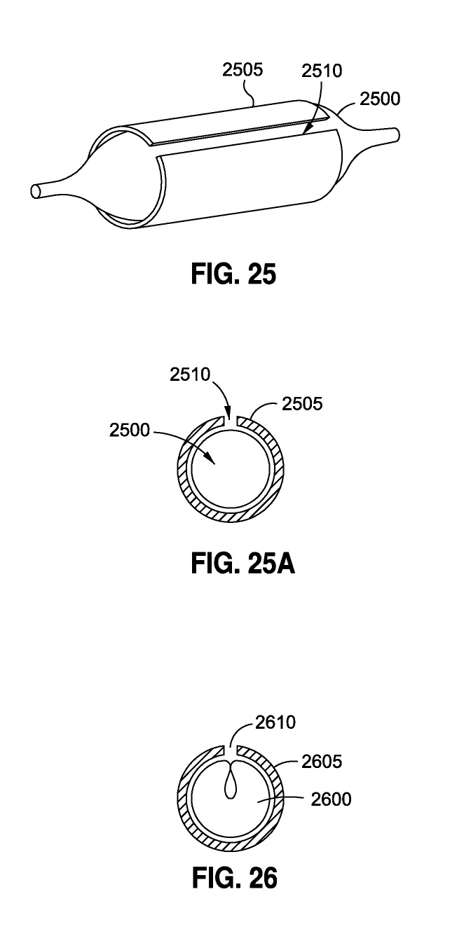

FIG. 25 illustrates a reservoir 2500 with an outer protective layer 2505. The outer protective layer 2505 may be an exoskeleton configured to wrap axially about the reservoir 2500. The outer protective layer 2505 may protect the reservoir 2500 from external forces. As shown, the outer protective layer 2505 may include a hinge cut 2510 causing a gap within the outer protective layer 2505. The hinge cut 2510 may allow the outer protective layer 2505 to be deformable, thereby allowing the reservoir 2500 to be compliant even after the fluid-volume level of the reservoir 2500 causes the outer diameter of the reservoir 2500 to exceed the inner diameter of the outer protective layer 2505. In other words, the hinge cut 2510 allows the outer protective layer 2505 to expand (increasing the width of the gap), which in turn allows the reservoir 2500 to continue to expand as it receives additional fluid. FIG. 25A is a cross-sectional view of the reservoir 2500 having the separate, overlaying outer protective layer 2505. In one embodiment, the reservoir 2500 may be attached or integrated with the outer protective layer 2505 (e.g., by using an adhesive). The outer protective layer 2505 may further be used with reservoirs of different shapes. For example, FIG. 26 illustrates a cross sectional view of an outer protective layer 2605 surrounding a u-shaped reservoir 2600. As shown, the outer protective layer 2605 may include an optional hinge cut 2610.

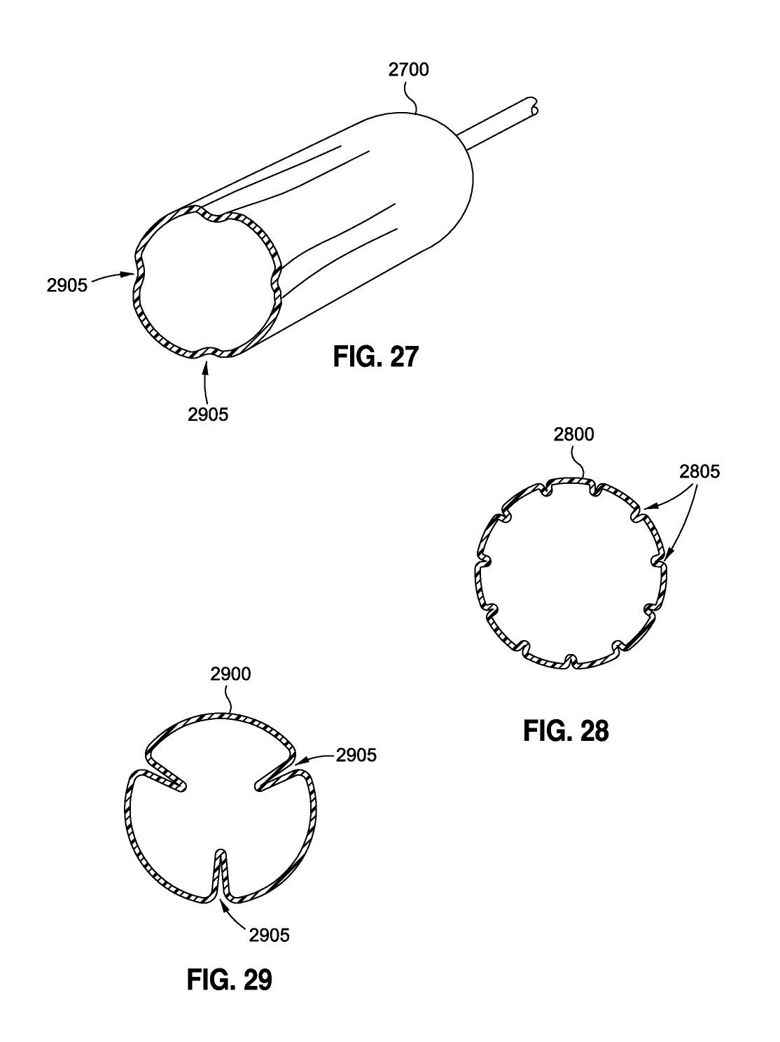

As discussed herein, the reservoirs (e.g., reservoir 2500 of FIG. 25 and reservoir 2600 of FIG. 26, among other embodiments of reservoirs) may be shaped differently. In addition and/or alternatively, the reservoirs may include depressions, pleatings or longitudinal structures along an outer circumferential perimeter. For example, FIG. 27 illustrates a reservoir 2700 having four patterned depressions 2705 which may buckle (inward) in a reproducible manner when suction is applied to the reservoir 2700 by a surgical trocar (not shown). However, other depression patterns are also possible. For example, FIG. 28 illustrates a cross section of a reservoir 2800 having eleven patterned depressions 2805. FIG. 29 illustrates a cross section of a reservoir 2900 having three patterned depressions 2905. While FIGS. 27-29 illustrate reservoirs 2700, 2800 and 2900 having equally spaced depressions of different depths, non-uniformly spaced depressions are also possible for the purpose of guiding deflation.

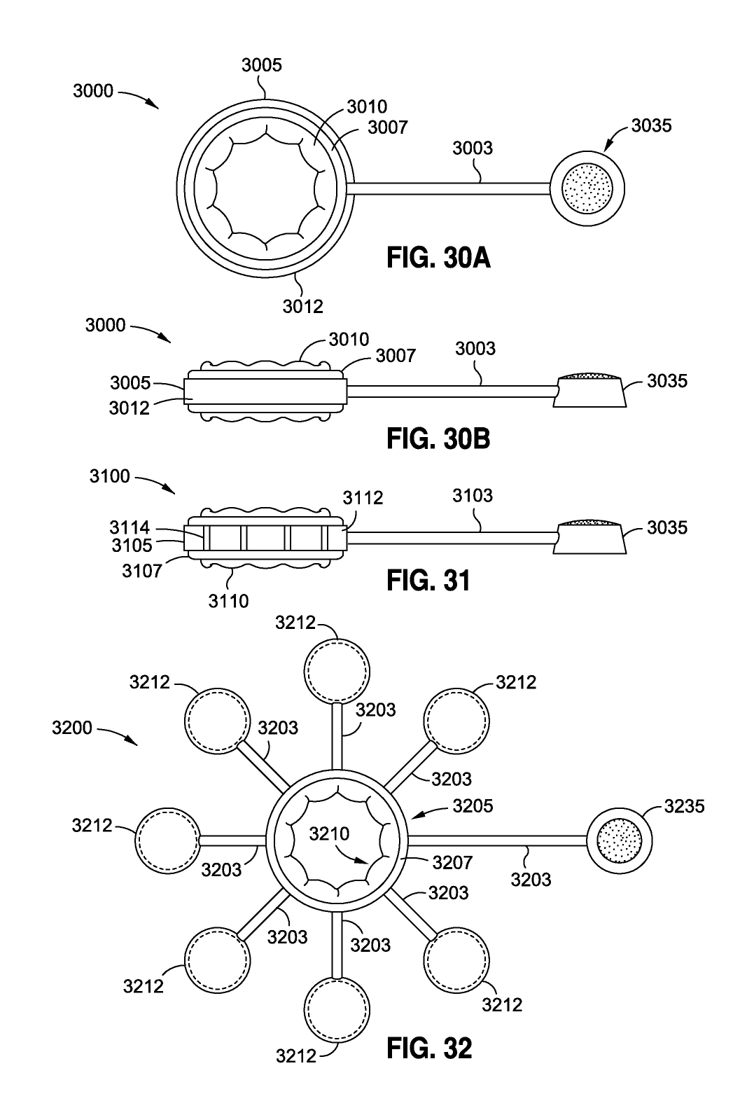

In addition and/or as an alternative to different reservoir shapes, a reservoir may be oriented in different ways. FIG. 30A illustrates one embodiment of a compliant reservoir system 3000 which may include a gastric band 3005 having an inflatable portion 3010, a ring 3007 and a compliant portion 3012. The gastric band 3005 may be fluidly coupled to a tubing 3003 and an access port 3035. As shown, the compliant reservoir 3012 may be oriented circumferentially about the ring 3007. The compliant reservoir 3012 may be in fluid communication (not shown) with the inflatable portion 3010 through a hole or other path (e.g., extending across the ring 3007). Accordingly, when a large bolus is swallowed by the patient, the fluid within the inflatable portion 3010 may be dispersed and may flow into the compliant reservoir 3012, thereby allowing the large bolus to pass through. Similar to the reservoirs 910, 1010, 1110 and 1250 of FIGS. 9-12, the compliant reservoir 3012 may be constructed of biocompatible, elastomeric material. FIG. 30B is a side view of the compliant reservoir system 3000 of FIG. 30A.

Similar to the compliant reservoir system 3000, a compliant reservoir system 3100 may include a gastric band 3105 having an inflatable portion 3110, a ring 3107 and a compliant portion 3112. The gastric band 3105 may be fluidly coupled to a tubing 3103 and an access port 3135. However, as shown in FIG. 31, the compliant reservoir system 3100 may further include vertical molding portions 3114. The vertical molding portions 3114 may be equally spaced apart about the outer circumference of compliant reservoir 3112 and may, in one embodiment, be attached to the ring 3107, functioning to provide the compliant reservoir 3112 with structural support. In this manner, the compliant reservoir 3112 may be integrated with the ring 3107.

FIG. 32 illustrates a compliant reservoir system 3200 having a gastric band 3205 with an inflatable portion 3210 and a ring 3207. The gastric band 3205 may in fluid communication with an access port 3235 and a plurality of compliant reservoirs 3212. As shown, seven reservoirs 3212 may be oriented radially around the gastric band 3205 and may be coupled to the gastric band 3205 via corresponding tubing 3203. While each reservoir 3212 is shown to be substantially equidistant from the gastric band 3205, alternative geometries are possible (e.g., one or more of the reservoirs 3212 may be closer to the gastric band 3205 via shorting tubing). In addition, the number of reservoirs 3212 may vary (e.g., any number of reservoirs 3212 between one and twenty inclusive, may be included). These reservoirs 3212 may function similarly as the reservoirs 3012 and 3112 of FIGS. 30A and 31, respectively. For example, when a large bolus is swallowed by the patient, the fluid within the inflatable portion 3210 may be dispersed and may flow into any or all of the compliant reservoirs 3212, thereby allowing the large bolus to pass through. In addition, the reservoir 3212 may be constructed out of any one or a combination of biocompatible, elastomeric material.

Reservoirs of different types, configurations, and orientations having been discussed, attention will now be turned to the fill substance of the gastric banding systems. The fill substances discussed herein may be applicable to any gastric banding system, including any of the compliant reservoir systems discussed above (e.g., systems 900, 1000, 1100, and 1200).

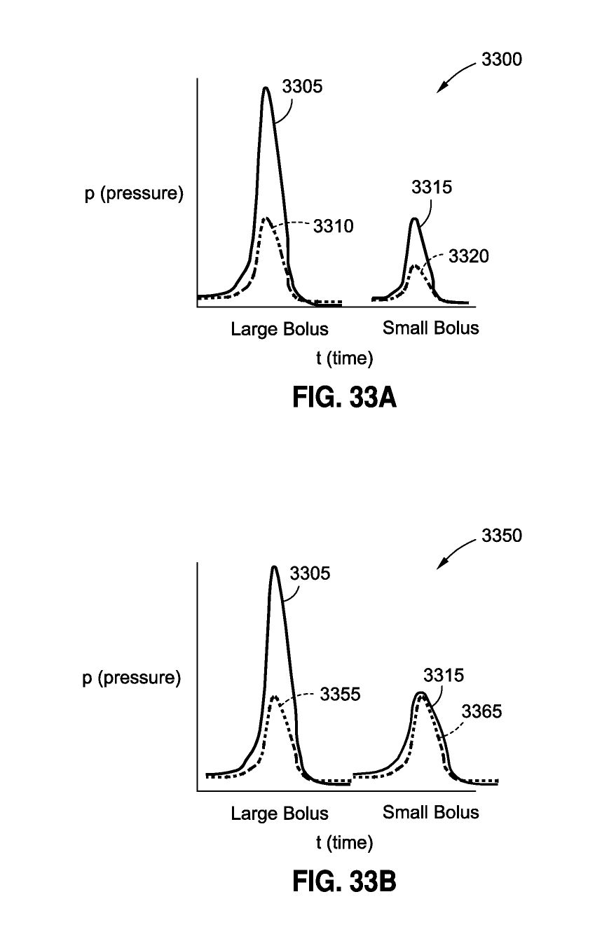

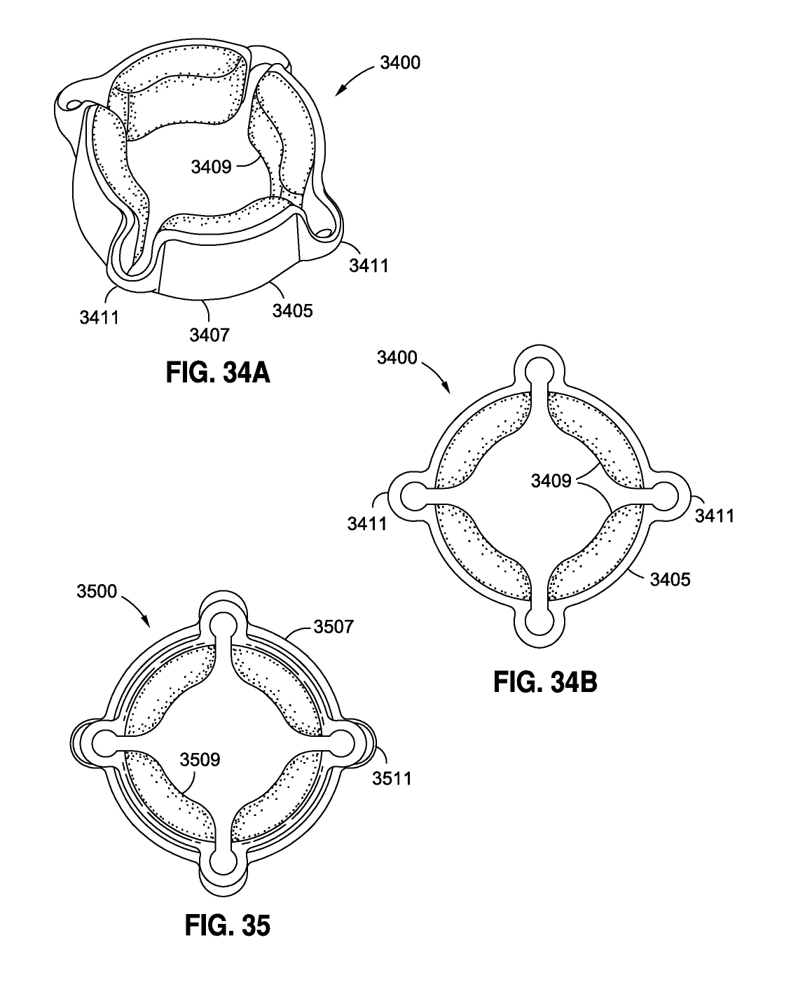

FIG. 33A illustrates a pressure-time graph 3300 comparing a compliant reservoir system utilizing saline or another fluid with a non-compliant gastric banding system. As shown, when a large bolus passes through the constriction of a non-compliant gastric banding system, a large spike in pressure 3305 is exerted whereas the same large bolus passing through the constriction of a compliant reservoir system utilizing saline may result in a lower pressure spike 3310. In addition, a higher pressure spike 3315 may occur when a small bolus passes through the constriction of the non-compliant gastric banding system, and a lower pressure spike 3320 may occur when the same small bolus passes through the constriction of the compliant reservoir system utilizing saline.

While FIG. 33A illustrates the benefits of a compliant reservoir system utilizing saline for situations where a large bolus is present over a non-compliant gastric banding system, further improvement may be possible with respect to small bolus reactions.

FIG. 33B illustrates a pressure-time graph 3350. The pressure-time spikes 3305 and 3315 correspond to a large bolus and a small bolus, respectively, passing through a constriction formed by a non-compliant gastric banding system. In comparison, the pressure spikes 3355 and 3365 correspond to a large bolus and a small bolus, respectively, passing through a constriction formed by a compliant reservoir system utilizing non-saline substances. More particularly, when a large bolus is introduced, the compliant reservoir system utilizing non-saline substances may perform substantially similar to a compliant reservoir system utilizing saline substances (e.g., comparing pressure spike 3310 of FIG. 33A and pressure spike 3355 of FIG. 33B). However, when a small bolus is introduced, the compliant reservoir system utilizing non-saline substances may perform substantially similar to a non-compliant reservoir system (e.g., comparing pressure spike 3315 of FIG. 33A and pressure spike 3365 of FIG. 33B). The response to large boluses and small boluses as illustrated in pressure spike 3355 and 3365, respectively, may be preferable for providing overall improved efficacy.

These non-saline substances which provide for the preferred pressure spikes 3315 and 3365 of FIG. 33B may include a pseudoplastic fluid, a Bingham plastic, and the like.

In one embodiment, a pseudoplastic fluid may be utilized to fill a gastric banding system (e.g., gastric banding system 500, 600). A pseudoplastic fluid may exhibit a decrease in viscosity under increases in the shear rate. Accordingly, only a slight increase in pressure would lead to relatively low shear rates (and relatively high viscosity) while a more substantial increase in pressure would lead to relatively high shear rates (and relatively low viscosity). In this manner, small to medium increases in pressure within the gastric banding system (e.g., gastric banding system 500, 600) will yield results similar to existing non-compliant gastric banding systems, while under higher pressure spikes, the pseudoplastic fluid (due to its lower viscosity at higher pressures) would flow out of the inflatable portions and into a reservoir, curbing the intensity of the pressure spike. Such large pressure spike may occur, for example, when a large bolus is attempting to pass through a constriction of the non-compliant gastric banding system. Once the pressure minimizes and the baseline pressure in the gastric banding system is re-established, the pseudoplastic fluid may gradually return to the inflatable portions.

In one embodiment, a Bingham plastic may be utilized as the fluid within the gastric banding system (e.g., gastric banding systems 500, 600). The Bingham plastic may be a material which does not flow until a minimum yield stress within the fluid is reached. In other words, prior to reaching the minimum yield stress, the Bingham plastic acts as a solid. However, once the minimum yield stress is reached, the Bingham plastic acts a fluid and may flow from the inflatable portions (e.g., the inflatable portion 510, 610) to a fluidly-coupled reservoirs (e.g., the reservoir 514, 614). Once the pressure minimizes and the baseline pressure is re-established, the Bingham plastic may flow again and may return to the inflatable portions (e.g., inflatable portions 510, 610).

In addition to pseudoplastic fluid, the Bingham plastic and the like, other materials may be used to fill any gastric banding system. Moreover, other configurations, including addition or removing various components of the gastric banding system, may be desirable when other fill substances are considered.

FIG. 34A illustrates a perspective view of one embodiment of a gastric band system 3400 having a different fill material than saline. The gastric band system 3400 may form a circumference about an upper stomach region of a patient and may provide pressure on the patient's stomach to induce satiety. Here, the gastric band system 3400 may include a gastric band 3405 having an outer ring 3407, cushions 3409 and one or more hinges 3411. The cushions 3409 may be filled with a gel and/or other substances. Alternatively or in addition, the cushions 3409 may be low durometer foam pads (e.g., less than 40 durometers). The gastric band 3405 may be self-contained and might not involve fluid systems having components such as tubing and access ports. As shown, the gastric band 3405 may include four cushions 3409 separated by four hinges 3411. However, additional cushions 3409 and/or hinges 3411 may be added, or one or more cushions 3409 and/or hinges 3411 may be removed. The hinges 3411 and the outer ring 3407 may be constructed out of a plastic or other polymer having dimensional stability, low creep and relatively high modulus of elasticity such that it may operate to function as a torsion spring. For example, a PEEK or polysulfone material may be utilized, among other materials. Alternatively, the hinges 3411 may be constructed out of metal (e.g., stainless steel, titanium, etc.). In one or more embodiments, when the hinge 3411 is constructed out of a metal, a silicone rubber may encapsulate the hinge 3411. Regardless of the construction of the hinge 3411, each may function as a torsion spring generating a substantially constant force on the patient's stomach even when the inside diameter of the gastric band 3405 increases due to the passage of a large bolus of food. For example, as the radial expansion of the gastric band 3405 occurs as caused by the passing of the large bolus of food, an increase in the lever arm of the hinges 3411 partially opposes (and thus cancels out) the effect of the increasing deflection, thereby resulting in a constant force.

FIG. 34B illustrates a top view of the gastric banding system 3400 of FIG. 34A. As shown, in one embodiment, the hinges 3411 may be integrated with the outer ring 3407 and the cushions 3409 may have a length substantially spanning a non-hinge portion of the outer ring 3407. The gastric banding system 3400 of FIGS. 34A and 34B may include a tunable hoop spring rate and may be customized to have an initial diameter for a given patient.

FIG. 35 illustrates a gas-filled gastric banding system 3500. While similar in appearance to the gastric banding system 3400 of FIGS. 34A and 34B, and including features like one or more hinges 3511 and one or more ring portions 3507, one difference between the gas-filled gastric banding system 3500 and the gastric banding system 3400 is that the cushions 3509 may be constructed out of different materials and may be filled with one or more gases including carbon dioxide, nitrogen, among others. By utilizing the gas-filled cushions 3509, lower radial stiffness may be achieved, thereby reducing discomfort for the patient when a large bolus attempts to pass through the constriction. A suitable gas-impermeable membrane may be used in constructing the cushions 3509 to prevent the gas from leaking out. For example, a silicone rubber may be used. In addition, coatings to further prevent gas leakage such as a diamond-like carbon, titanium nitrite, parylene, aclar, among other gas impermeable substances, and combinations thereof may be applied to the silicone rubber. In one embodiment, the gastric banding system 3500 might not require a traditional access port for occasional adjustment. Instead, an adjustment just prior to implantation may provide a constant pressure configured to serve the constriction and/or the neural stimulation function properly while large boluses of food are able to pass through due to the compressibility of the gas-filled gastric banding system 3500.

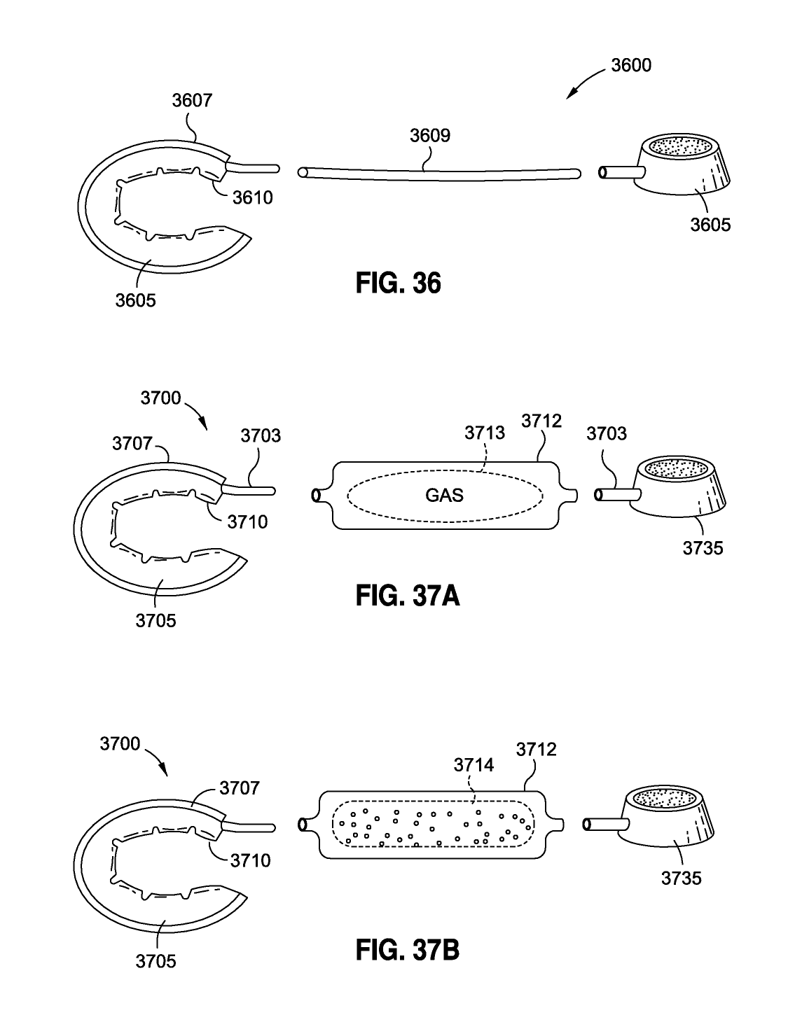

The use of gas as a fill substance is not limited to the embodiment of FIG. 35. For example, as shown in FIG. 36, a gastric banding system 3600 may include a gastric band 3605 having a ring 3607 and an inflatable portion 3610 connected to a tube 3609 and an access port 3635. However, the inflatable portion 3610, the tube 3609 and the access port 3635 may all be constructed out of gas impermeable substances such as silicone rubber and/or coated with gas impermeable substances including, but not limited to, a diamond-like carbon, titanium nitrite, parylene, aclar, among other gas impermeable substances and combinations of substances.

In a further embodiment, a hybrid gas-saline gastric banding system 3700 is illustrated in FIG. 37A. As shown, the hybrid gas-saline gastric banding system 3700 may include a gastric band 3705 having a ring 3707 and an inflatable portion 3710. The gastric band 3705 may be in fluid communication with a reservoir 3712 and an access port 3735 via tubing 3703. The gastric banding system 3700 may be filled with a standard saline solution. However, a further encapsulated member 3713 may also be present within the hybrid gas-saline gastric banding system 3700. The encapsulated member 3713 may be a balloon filled with a gas such as carbon dioxide, nitrogen, and the like. The encapsulated member 3713 may function to further provide flexibility and/or otherwise lower the radial stiffness within the hybrid gas-saline gastric banding system 3700 by moving from the inflatable portion 3710 to the reservoir 3712 when a large bolus is attempting to pass through the constriction of the gastric band 3705. In one embodiment, the encapsulated member 3713 is within the fluid path and may travel to any component of the gastric banding system 3700 in response to fluid movement through, e.g., the tubing 3703. In one embodiment, the encapsulated member 3713 may be configured to be located within or traverse back into the inflatable portion 3710 when a large bolus is not attempting to pass through (e.g., when the hybrid gas-saline gastric banding system 3700 is in an equilibrium state).

In one embodiment, the encapsulated member 3713 may be constructed out of silicone rubber and may be coated with one or more materials to enhance the ability of the silicone rubber to be gas impermeable thereby preventing the encapsulated member 3713 from leaking.

In one embodiment, the encapsulated member 3713 might not be designed to not fit through the tubing 3703 and traverse between the different components of the hybrid gas-saline gastric banding system 3700. In other words, the encapsulated member 3713 may permanently reside in any one of the components, e.g., the reservoir 3712.

FIG. 37B illustrates the hybrid gas-saline gastric banding system 3700 utilizing a different encapsulated member 3714 in place of the encapsulated member 3713 of FIG. 37A. The encapsulated member 3714 may have the functionality similar to the encapsulated member 3713 of FIG. 37A, but may be constructed out of a closed cell foam instead of gas.

Modifications may be further made to the hybrid gas-saline gastric banding system 3700. For example, other compliant portions may be added. Alternatively and/or in addition, in one embodiment, the hybrid gas-saline gastric banding system 3700 as discussed above with respect to FIGS. 37A and 37B may be further modified to eliminate the access port 3735 leaving only the gastric band 3705, the reservoir 3712 and one of the encapsulated members 3737 or 3738 (not shown). Eliminating the access port 3735 renders the hybrid gas-saline gastric banding system 3700 self-adjusting and may provide constant pressure on the stoma of the patient.

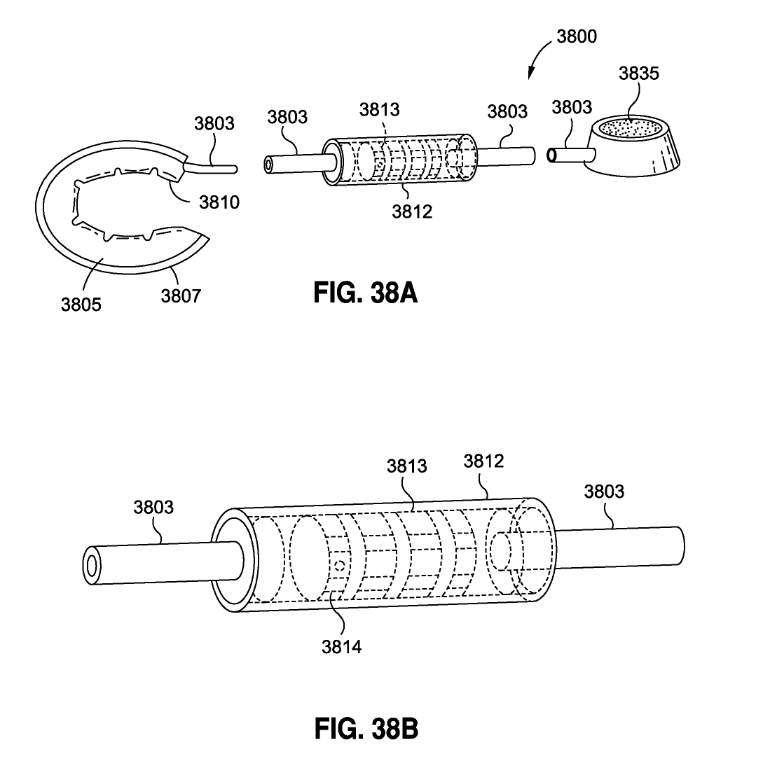

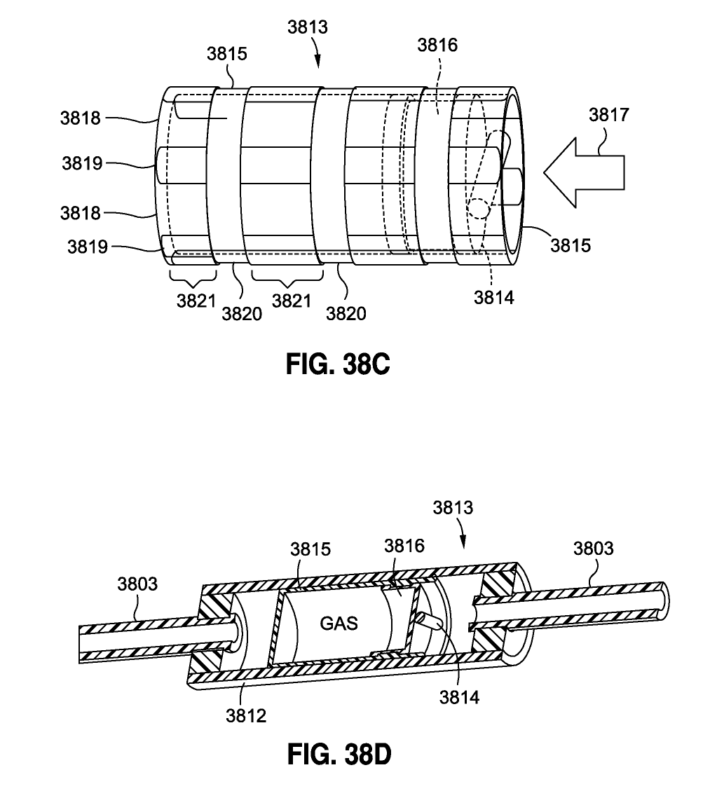

FIG. 38A illustrates another embodiment of a hybrid gas-saline gastric banding system 3800. As shown, the hybrid gas-saline gastric banding system 3800 may include a gastric band 3805 having a ring 3807 and an inflatable portion 3810. The gastric band 3805 may be in fluid communication with a reservoir 3812 and an access port 3835 via tubing 3803. The gastric banding system 3800 may be filled with a standard saline solution. The reservoir 3812 may be cylindrical in shape and may be constructed out of a silicon rubber or another durable, gas impermeable material. The tubing 3803 may be held in place within the reservoir 3812 by an adhesive layer or patch. In addition, the reservoir 3812 may house a gas spring 3813. In one embodiment, the gas spring 3813 may be retained in the reservoir 3813 by radial interference (e.g., the gas spring 3813 may be designed to fit tightly within the reservoir 3812 to avoid slippage or other un-intended movement). In one embodiment, the gas spring 3813, while being movably fixed within the reservoir 3812, may still allow for fluid to pass through by having uneven surfaces (e.g., non-circular geometry which creates gaps and/or openings for fluid to pass through between an outside surface of the gas spring 3813 and an inner diameter of the reservoir 3812). Generally, the gas spring 3813 may operate to provide volume flexibility and/or low radial stiffness in the gastric banding system 3800. For example, when a large bolus of food passes through a constriction of the gastric band 3805, pressure caused by the bolus on the inflatable portion 3810 may cause a transfer of fluid from the inflatable portion 3810 to the reservoir 3812. One function of the gas spring 3813 is to alleviate the pressure spike caused by the food bolus by moving a piston within the gas spring 3813 and winding the gas spring 3813. Once the pressure is removed (e.g., the bolus having been passed through the constriction), the gas spring 3813 may unwind and revert back to a non-compressed orientation.

FIG. 38B illustrates the reservoir 3812 and the gas spring 3813 apart from the other portions of the hybrid gas-saline gastric banding system 3800. FIG. 38C illustrates a further deconstructed view of the gas spring 3813 apart from the reservoir 3812. As shown in FIG. 38C, a pin 3814 may be fixed in place to the inner diameter of the gas spring housing 3815 thereby limiting the movement of the piston 3816. Arrow 3817 illustrates a direction of fluid pressure (e.g., caused by a large bolus passing through the constriction of the gastric band 3805) which may move the piston 3816 and compress the gas within the gas spring housing 3815. The gas within the gas spring housing 3815 may be sealed between the piston 3816 and compressed when pressure is introduced. In one embodiment, the piston 3816 and the inner surface of the gas spring housing 3815 may be formed out of materials with very low coefficient of friction such that the overall friction force does not overly prevent the piston 3816 from moving when a fluid pressure is introduced. In one embodiment, the outside surface of the gas spring housing 3815 may include contact segments 3821 and fluid passageway segments 3820 configured in an alternating manner. An outer diameter of any given fluid passageway segment 3820 may be smaller than an outer diameter of any given contact segment 3821. The contact segments 3821 may further include curved portions 3818 for contacting an inner diameter of the reservoir 3812 of FIG. 38B, thereby holding or fixing the gas spring 3813 in place. The contact segments 3821 may further include flat portions 3819 which might not contact the inner diameter of the reservoir 3812, thereby allowing fluid flowing through the fluid passageway segment portions 3818 to also flow through the contact segments 3821. In this manner, fluid within the gastric banding system 3800 may flow from, for example, the gastric band 3805 to the access port 3835, and vice versa. In one embodiment, the flat portions 3819 and the curved portions 3813 of each contact segment 3821 may alternate in configuration around the circumference of the gas spring housing 3815.

FIG. 38D is a cross-sectional view of the reservoir 3812 and the gas spring 3813 apart from the other portions of the hybrid gas-saline gastric banding system 3800 to better illustrate the configuration of the components therein. Appropriate materials to construct one or more parts of the gas spring 3813 (e.g., gas spring housing 3815, piston 3816, etc.) may include gas-impermeable and/or saline-impermeable substances such as plastics like PTFE.

In addition to and/or as an alternative to a hybrid gas-saline gastric banding system of FIG. 38 having a reservoir (e.g., the reservoir 3812) functioning as the gas-saline component, other components of the gastric banding system may be the gas-saline component. For example, an access port of any gastric banding system may be the gas-saline component.

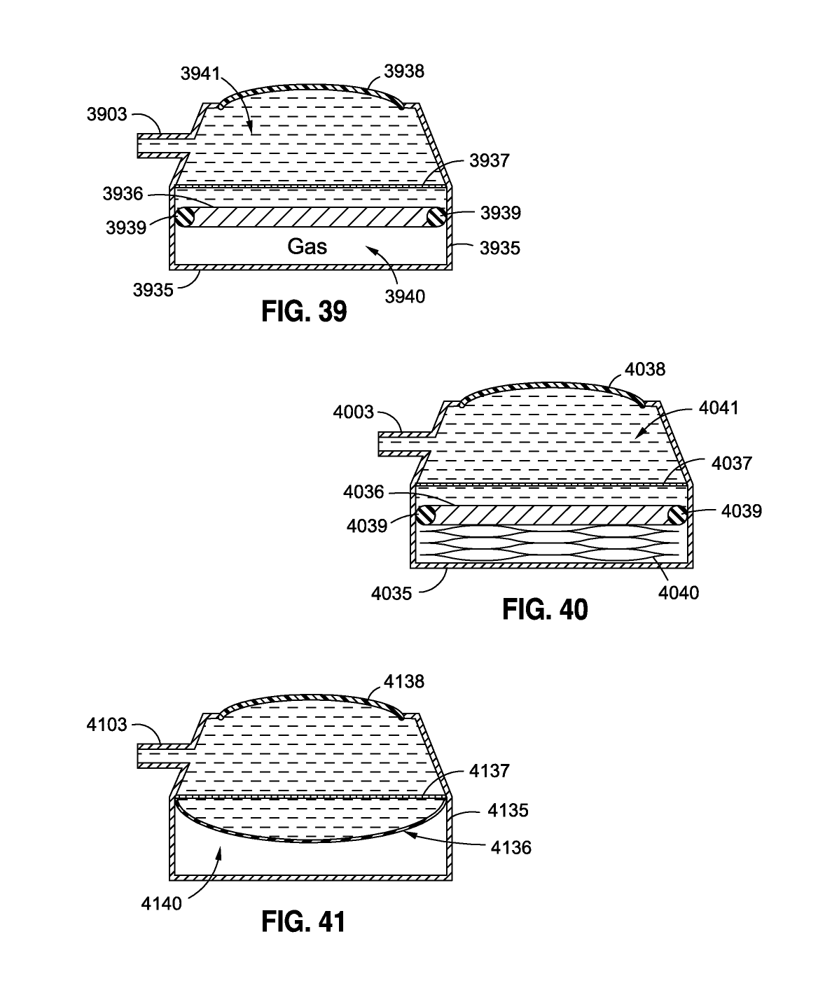

FIGS. 39-41 illustrate various embodiments of access ports 3935, 4035, 4135. While the rest of the gastric banding system is not shown in these figures, the access ports 3935, 4035, 4135 may be in fluid communication with other components of the gastric banding system (e.g., a gastric band and/or a reservoir) via tubes 3903, 4003, 4103, respectively. For example, access ports 3935, 4003, 4103 may each be the access port 435 or 535.

Turning to FIG. 39, in one embodiment, the access port 3935 may include a movable surface 3936 which may move in response to a pressure change within the gastric banding system. For example, the movable surface 3936 may be a complaint portion that moves to increase the volume of the fluid portion 3941 (and therefore increases compliance) when the pressure increases within the gastric banding system (e.g., in response to a large bolus moving through a constriction of the gastric band). Once the pressure is reduced (e.g., the large bolus passing through the constriction), the movable surface 3936 may return to its original position, thereby decreasing the volume of the fluid portion 3941 of the access port 3935. The access port 3935 may further include a septum 3938, a fluid-permeable membrane 3937, o-rings 3939 and a gas spring portion 3940 filled with a gas 3935.

Alternatively, the gas spring portion 3940 may be replaced with a wave spring, a cantilever spring, a constant force spring, a coil spring, a leaf spring, a Belleville spring, a hybrid polymer coil-air spring and the like. In addition, a vacuum (not shown) may be incorporated in order to achieve the desired pressure response.

FIG. 40 illustrates an example of an access port 4035 having a wave spring 4040. The access port 4035 may be in fluid communication with other components of the gastric banding system (e.g., a gastric band and/or a reservoir) via tube 4003. In one embodiment, the access port 4035 may include a movable surface 4036 which may move in response to a pressure change. For example, the movable surface 4036 may be a complaint portion that moves to increase the volume of the fluid portion 4041 (and therefore increases compliance) in response to a large bolus moving through a constriction of the gastric band (not shown). Once the pressure is reduced (e.g., the large bolus passes through the constriction), the movable surface 4036 may return to its original position thereby decreasing the volume of the fluid portion 4041 of the access port 4035. The access port 4035 may further include a septum 4038, a fluid-permeable membrane 4037, o-rings 4039 and a wave spring 4040.

In one embodiment, the spring (e.g., gas spring 3940 of FIG. 39 or wave spring 4040 of FIG. 40) may be removed as shown in FIG. 41. Here, the moving surface 4136 may comprise a flexible membrane that deforms when subjected to pressures above a certain threshold (which in one example, may be set to approximate the pressure induced when a large bolus is passing through a constriction). Once the pressure falls below the threshold again (e.g., when the large bolus passes through the constriction), the moving surface 4136 may return to its original configuration. The moving surface 4136 may be constructed out of silicone or another biocompatible flexible polymer, among other materials. The moving surface 4136 may be attached to the inner wall of the access port 4135 or may be integrated with a septum 4138 of the access port 4135.

Each of the access ports 3935, 4035 and 4135 may include a corresponding septum 3938, 4038 and 4138. The septum 3938, 4038 and 4138 may function to allow a penetrating needle to add or remove fluid thereby adjusting the total fluid volume within the corresponding gastric banding system. In addition, the access ports 3935, 4035 and 4135 may include a corresponding fluid-permeable membrane 3937, 4037 and 4137 having a plurality of small openings (not shown) which may be large enough to allow fluid to pass through, but are small enough to prevent the passage of the needle. The fluid-permeable membranes 3937, 4037 and 4137 may be constructed out of a metal, ceramic, carbon, polymer or any combination thereof. Other materials may also be used so long as the material prevents the passage of the needle. The fluid-permeable membranes 3937, 4037 and 4137 may be attached to the inner wall of the corresponding access port 3935, 4035 and 4135.

In addition, the access ports 3935 and/or 4035 may include a corresponding sealing ring 3939 and 4039 to prevent fluid from entering the spring portions 3940 and 4040. The sealing rings 3939 and 4039 may be a polymer o-ring, a metal seal ring, a ceramic seal ring, a polymer seal ring and the like.

In addition to using pressure to control the flow of fluid, gas, among other substances within a gastric banding system, specific restriction devices may also be utilized. For example, a one-way valve, a pressure relief valve, orifices, turbulence controllers, sponges, environment-adapting devices, among other restriction devices may control a rate at which a component (e.g., a compliant reservoir) may receive fluid from another component (e.g., a gastric band).

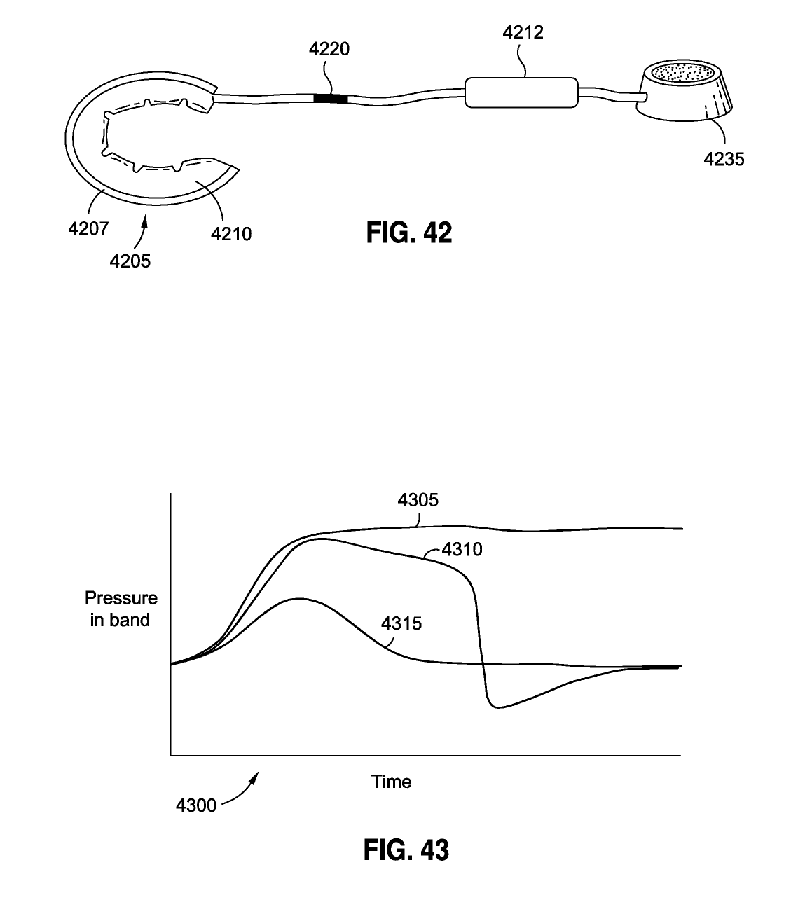

FIG. 42 illustrates an embodiment of a gastric banding system 4200 having a gastric band 4205, an inflatable portion 4210, and a ring 4207 in fluid communication with a reservoir 4212 through a restriction device 4220. Also shown is an access port 4235 in fluid communication with the above-mentioned components of the gastric banding system 4200. The restriction device 4220 may be electronically controlled, modulated with an external adjustment system or may be a passive restriction device configured to be independent from being controlled by an external tool or device.

In practice, any one (or more) of a plurality of restriction devices (e.g., restriction device 4220) may be used within the fluid path of the gastric banding system 4200 or any other gastric banding system. Further, additional restriction devices may be added to achieve the desired flow rates. FIG. 43 illustrates a graph 4300 depicting how the time-pressure curve may change when such a restriction device 4220 is used (e.g., an orifice having an inner diameter which limits the flow rate of the fluid in both directions) in one embodiment. For comparison, line 4305 is a time-pressure curve corresponding to a standard non-compliant band. As shown, the pressure may sharply rise when a large bolus encounters the constriction formed by the standard non-compliant band may become obstructed. As shown, the pressure may remain high for the duration of the obstruction caused by the bolus, and the patient may experience discomfort for a long period of time. Line 4315 is a time-pressure curve corresponding to a compliant band without a restriction device. As shown, the large bolus of food may cause only a modest increase in pressure (and hence, lesser patient discomfort) as the bolus passes through a constriction formed by the compliant band without the restriction device. This illustrates a potential drawback in the compliant band without a restriction device as the patient might not be aware that he or she has eaten an inappropriately large piece of food.

Line 4310 is a time-pressure curve corresponding to a compliant band with a restriction device (e.g., as shown in FIG. 42). As shown by the line 4310, the pressure may rise quickly causing the patient to feel pressure for a short period of time as the fluid drains slowly from the band (e.g., the gastric band 4205) and into the reservoir (e.g., the reservoir 4212). After a short period of time (which may serve as a reminder to the patient to modify his or her eating pattern and to avoid eating large pieces of food) the band drains enough fluid into the reservoir to allow the bolus to pass through, thereby returning the pressure near equilibrium.

Balloons, reservoirs, access ports and flow control devices having been discussed, attention will now be turned to the gastric band portion of a gastric banding system.

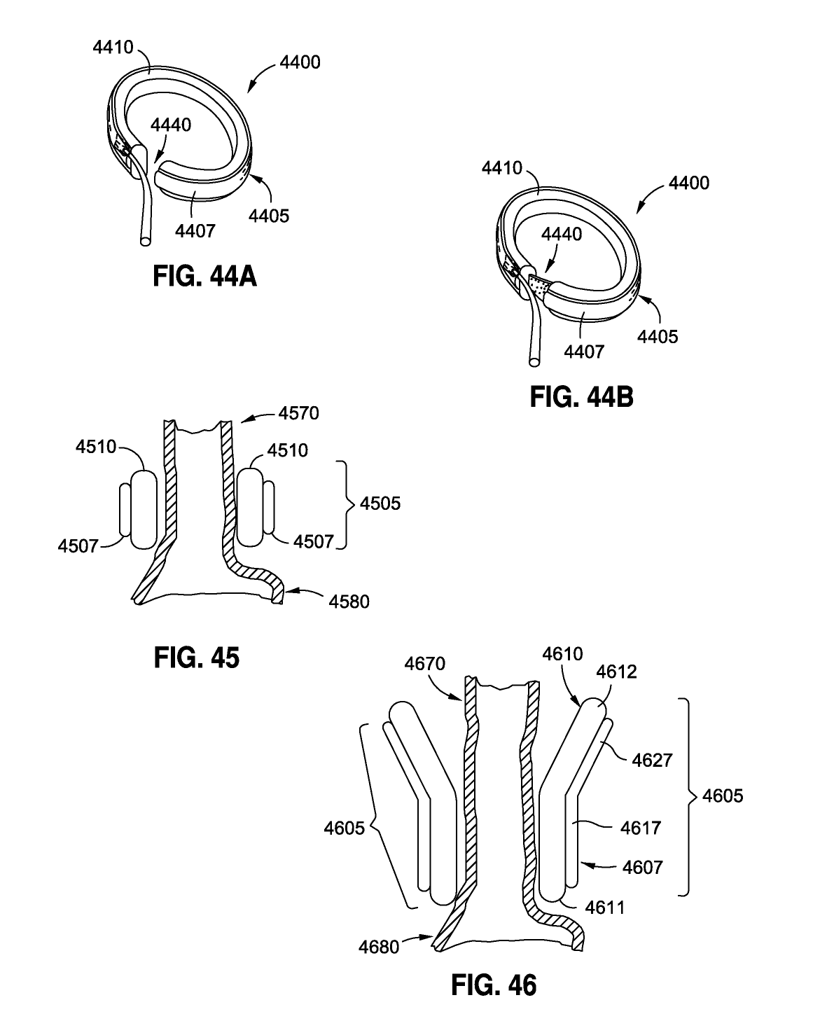

FIG. 44A illustrates an embodiment of an always-open gastric banding system 4400 including a gastric band 4405 having a ring 4407 and an inflatable portion 4410. Instead of having a flexibly-stiff ring locked in place at an open end (as traditionally utilized in a standard gastric band), the gastric band 4405 does not include a locking portion and replaces the flexibly-stiff ring with a more inflexible ring (e.g., ring 4407) such as a snap ring, a split ring, retaining ring and the like. The more inflexible ring (e.g., the ring 4407) may be constructed out of metal and a corresponding spring rate may be adjusted to provide a substantially constant force on the outside of the patient's stomach. In one embodiment, the adjustability of the inflatable portion (e.g., the inflatable portions 4410) may be eliminated. As shown, in this embodiment of FIG. 44, the other components (access port, tubing, etc.) of a standard gastric band system may be removed. In operation, once the gastric banding system 4400 is implanted, the gastric band 4405 may flex open when a large bolus moves through the constriction of the gastric band 4405 on the stomach region. Once the large bolus passes the constriction of the gastric band 4405, the gastric band 4405 may return to its original orientation. As shown, the gastric banding system 4400 may include a variable width gap 4440 between the opposite ends of the gastric band 4405. The gap 4440 may serve to provide a more effective response to a patient's anatomy and physiological conditions (e.g., swallowing a large bolus).

FIG. 44B illustrates another embodiment of the gastric banding system 4400 of FIG. 44A. As shown, the gastric banding system 4400 further includes elastic members 4450, which may be tethered to “close” the variable width gap 4440. In this embodiment, the elastic members 4450 may be constructed out of a flexible polymer and may function to enhance the ability of the gastric banding system 4400 to remain in place.

In addition to and/or as an alternative to gastric banding systems having an “open” configuration (e.g., the gastric banding system 4400), a gastric band may also be configured to have portions of various sizes. FIG. 45 illustrates a cross-sectional view of the standard gastric band 4505 having a ring 4507 providing structural support to an inflatable portion 4510. As shown, the standard gastric band 4505 is placed or fixed about the esophageal-gastric junction between a patient's esophagus 4570 and stomach 4580.

FIG. 46 illustrates an embodiment with a wider section. As shown in the cross-sectional view of FIG. 46, the improved gastric band 4605 may still include a ring 4607 and an inflatable portion 4610. However, the design of the ring 4607 may include a standard portion 4617 and a wider portion 4627, and the inflatable portion 4610 may include a corresponding standard portion 4611 and a corresponding wider portion 4612. The wider portions 4612 and 4627 may be placed orad (closer or toward the mouth of the patient) as compared to the standard portions 4611 and 4617. The wider portions 4612 and 4627 may operate to stimulate and restrict the patient's esophageal-gastric junction when the patient is not eating or swallowing small boluses. When the patient swallows medium-sized boluses, the wider portions 4612 and 4627 may channel the medium bolus through the standard portions 4611 and 4617. And when the patient swallows large boluses, the wider portions 4612 and 4627 may function to relieve the stress on the patient's tissue and assist to prevent formations of pouch dilatations. The wider portions 4612 and 4627, as shown in FIG. 46, may increase in diameter in the orad direction. Optional suture tabs (not shown) may also be added to the gastric band 4605, for example, when the additional width of the wider portions 4612 and 4627 render it preferable that a standard gastro-gastric suturing process is not used. In one embodiment, the wider portions 4612 and 4627 may be adjustable.

In one embodiment, the wider portions 4612 and 4627 may have the same properties as the standard portions 4611 and 4617 (e.g., same materials, durometer, balloon-to-ring width ratio). Additionally, and/or as an alternative, the height of the ring 4607 and/or the height of the inflatable portion 4610 may be adjustable and might not span the entire height of the gastric band 4600 (not shown).

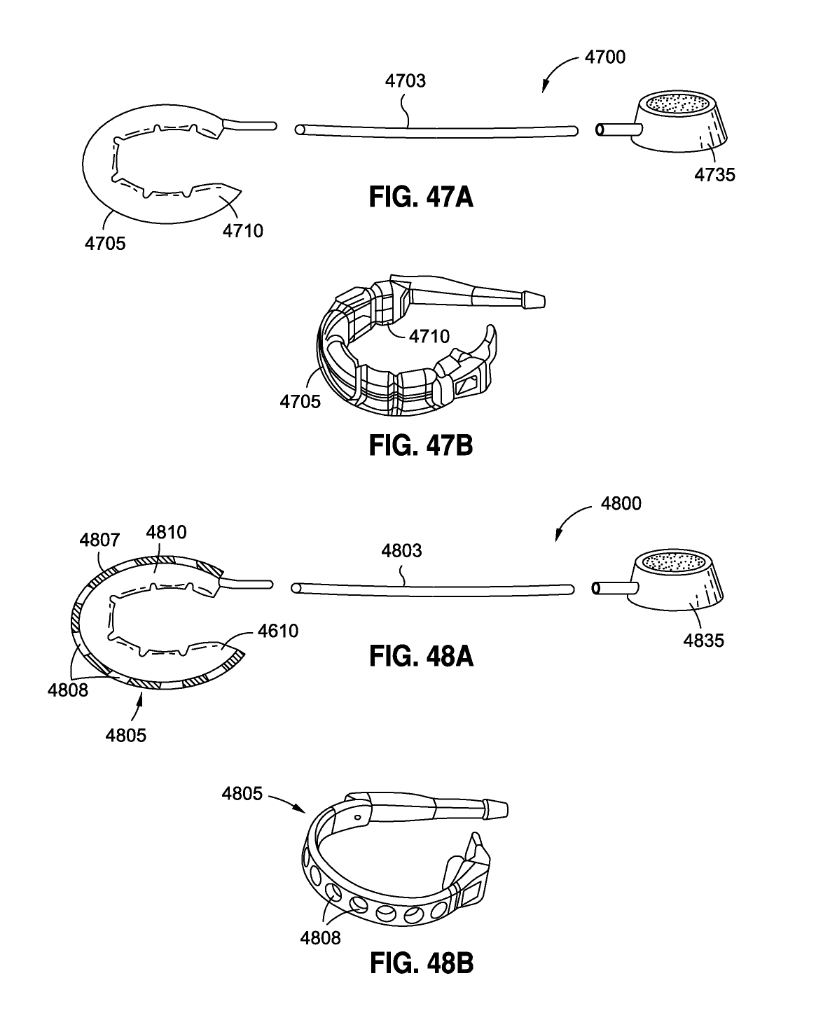

Further configurations of a gastric band system may include alterations to the ring portion. For example, FIG. 47A illustrates a gastric banding system 4700 having a gastric band 4705, a tube 4703 and an access port 4735. As shown, the gastric band 4705 may include inflatable portion 4710 without the presence of a ring. By eliminating the ring, the gastric banding system 4700 may be more compliant. As shown, the gastric banding system 4700 may rely on the inflatable portion 4710 alone for ring structure. FIG. 47B illustrates a perspective view of the gastric band 4705 without the ring structure, and with the other structures of the gastric banding system 4700 omitted.

FIG. 48A illustrates another embodiment of a gastric banding system 4800 having a gastric band 4805, a tube 4803 and an access port 4835. As shown, the gastric band 4805 may include inflatable portion 4810 and modified ring 4807 having holed portions 4808. The addition of the holed portions 4808 may increase the compliance of the gastric banding system 4800. FIG. 48B illustrates a perspective view of the gastric band 4805 with holed portions 4808. The inflatable portion 4810 and the other structures of the gastric banding system 4800 have been omitted for clarity.

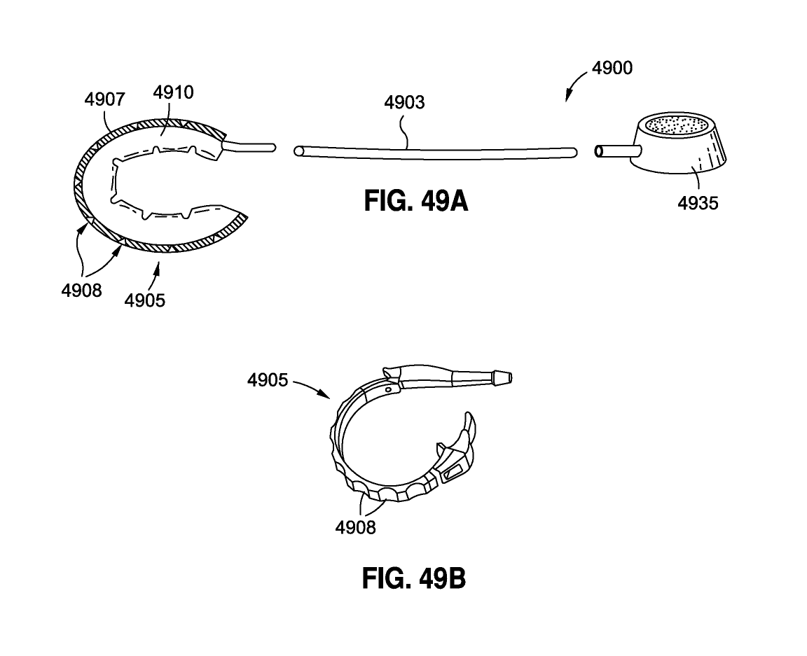

FIG. 49A illustrates another embodiment of a gastric banding system 4900 having a gastric band 4905, a tube 4903 and an access port 4935. As shown, the gastric band 4905 may include an inflatable portion 4910 and modified ring 4907 having cut-out or tapered portions 4908. The cut-out or tapered portions 4908 may increase the compliance of the gastric banding system 4900. FIG. 49B illustrates a perspective view of the gastric band 4905 with the cut-out or tapered portions 4908. The inflatable portion 4910 and the other structures of the gastric banding system 4800 have been omitted for clarity.

The gastric bands 4705, 4805 and 4905 may be more flexible (e.g., by having decreased ring stiffness) than a standard gastric band, thereby resulting in gastric banding systems 4700, 4800 and 4900, respectively, having muted pressure or force spikes on the tissues (e.g., in the esophageal-gastric junction) in a patient when the patient consumes a large bolus of food. Furthermore, each of the gastric bands 4705, 4805 and 4905 may have different, configurable torsional and ring stiffness characteristics to further create an ergonomically ideal function to serve the patient.

Furthermore, although various compliant components are illustrated in one or more of the figures, it should be understood that any combination of the various compliant components may be utilized in different embodiments. For example, an embodiment may include one compliant component (e.g., only the ring, the tubing, or the access port may be compliant). In other embodiments, any combination of the ring, the tubing, and the access port may be compliant. For example, an embodiment may include a compliant ring and a compliant port, an embodiment may include compliant tubing and a compliant port, or an embodiment may include a compliant ring and compliant tubing. Any combination of compliant components is within the scope of the present invention.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A self-adjusting gastric banding system for the treatment of obesity that adjusts to allow a bolus to pass through a constriction in a patient's stomach formed by the self-adjusting gastric banding system, the self-adjusting gastric banding system comprising:
a gastric band having an inflatable portion disposed about the stomach of the patient, the inflatable portion capable of constricting the stomach;
an access port fluidly coupled to the inflatable portion, the access port capable of receiving a fluid to inflate, or for removing a fluid to deflate, the inflatable portion of the gastric band;
a tubing coupled to the gastric band and the access port, the tubing carrying fluid between the inflatable portion of the gastric band and the access port; and
a compliant reservoir attached to at least a portion of the tubing and configured to receive fluid from the inflatable portion of the gastric band for automatically relaxing the constriction formed by the inflatable portion of the gastric band sufficiently to allow the bolus to pass through the constriction.

2. The self-adjusting gastric banding system of claim 1 wherein the tubing traverses through an interior cavity of the compliant reservoir.

3. The self-adjusting gastric banding system of claim 1 wherein the tubing includes an open portion, the entirety of the open portion located within the compliant reservoir, the open portion configured to allow fluid to flow into and out of the compliant reservoir.

4. The self-adjusting gastric banding system of claim 1 wherein the tubing includes a portion having a plurality of openings, and each of the openings fully located within the compliant reservoir for allow fluid to flow into and out of the compliant reservoir.

5. The self-adjusting gastric banding system of claim 1 wherein the compliant reservoir is a tube-shaped cylinder having a star-shaped structure defining an outer circumference, the star-shaped structure being deformable in response to an increase in fluid volume in the compliant reservoir.

6. The self-adjusting gastric banding system of claim 1 wherein the compliant reservoir is a tube-shaped cylinder configured to expand in diameter at a mid-point of the cylinder in response to an increase in fluid volume in the compliant reservoir.

7. The self-adjusting gastric banding system of claim 1 wherein the compliant reservoir is a flattened tube configured to expand in response to an increase in fluid volume in the compliant reservoir.

8. The self-adjusting gastric banding system of claim 7 wherein the flattened tube further includes indented portions which expand in response to the increase in fluid volume in the compliant reservoir.

9. The self-adjusting gastric banding system of claim 1 wherein the compliant reservoir is a u-shaped tube having two u-shaped portions, the u-shaped tube configured to expand in response to a first increase in fluid volume in the compliant reservoir such that one of the two u-shaped portions becomes flat, while the other of the two u-shaped portions remains u-shaped, the u-shaped tube configured to expand in response to a second increase in fluid volume in the complaint reservoir such that the u-shaped tube is transformed into a substantially oval tube.

10. The self-adjusting gastric banding system of claim 1 wherein the compliant reservoir includes a compliant tube and an exterior shell, the exterior shell configured to circumferentially surround the compliant tube while leaving a gap between an outer surface of the compliant tube and an inner surface of the exterior shell.

11. The self-adjusting gastric banding system of claim 1 wherein the compliant reservoir comprises a plurality of coiled wings, each of the coiled wings having a preset stress and being configured to uncoil and straighten out in response to an increase in fluid within the compliant reservoir.

12. The self-adjusting gastric banding system of claim 1 wherein the compliant reservoir may include an internal spring configured to prevent the compliant reservoir from being kinked, bent or compressed disrupting a flow of fluid through the compliant reservoir.

13. The self-adjusting gastric banding system of claim 1 wherein the compliant reservoir may include an internal cage configured to prevent the compliant reservoir from being kinked, bent or compressed disrupting a flow of fluid through the compliant reservoir.

14. The self-adjusting gastric banding system of claim 1 wherein the compliant reservoir may include a plurality of internal, parallel rings configured to prevent the compliant reservoir from being kinked, bent or compressed disrupting a flow of fluid through the compliant reservoir.

15. The self-adjusting gastric banding system of claim 1 wherein the compliant reservoir may include an external structure wrapping axially about the compliant reservoir, the external structure being deformable in response to the compliant reservoir having a fluid volume exceeding a threshold, the external structure further having a hinge cut.

16. The self-adjusting gastric banding system of claim 1 wherein the compliant reservoir may include depressions along an outer circumferential perimeter, the depressions configured to buckle inward in a reproducible manner when suction is applied to the compliant reservoir.

17. The self-adjusting gastric banding system of claim 1 further comprising a ring configured to surround an entirety of the inflatable portion, further wherein the compliant reservoir surrounds an outer perimeter of the ring.

18. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
an access port fluidly coupled to the inflatable portion to fill and drain the inflatable portion;
a tubing coupled to the inflatable portion and the access port, the tubing for carrying fluid between the inflatable portion and the access port; and
a compliant reservoir coupled to the access port at a first end and being closed at a second end, the compliant reservoir for automatically relaxing the constriction formed by the self-adjusting gastric band and allowing the large bolus to pass through the constriction.

19. The self-adjusting gastric band of claim 18 wherein the compliant reservoir is a coiled tube having a preset stress, the coiled tube configured to uncoil and straighten out in response to an increase in fluid within the compliant reservoir.

20. The self-adjusting gastric band system of claim 18 wherein the compliant reservoir is tube having a bulged reservoir at the second end, the bulged reservoir configured to enlarge in response to an increase in fluid within the compliant reservoir.

21. A self-adjusting gastric band that adjusts to allow a large bolus of food to pass through a constriction in a patient's stomach formed by the self-adjusting gastric band, the self-adjusting gastric band comprising:
an inflatable member configured to inflate when fluid is introduced into the inflatable member and deflate when fluid is removed from the inflatable member, the inflatable member configured to be situated about an esophageal-gastric junction of the patient;
an access port fluidly coupled to the inflatable portion to inflate and deflate the inflatable portion; and
a plurality of compliant reservoirs radially oriented about the inflatable portion, and fluidly coupled to the inflatable portion and the access port, the plurality of compliant reservoirs for automatically relaxing the constriction formed by the self-adjusting gastric band and allowing the large bolus to pass through the constriction by receiving fluid from the inflatable portion.

22. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient, the inflatable portion configured to be filled with a pseudoplastic fluid or a Bingham plastic;
an access port coupled to the inflatable portion to fill and drain the inflatable portion of the pseudoplastic fluid or the Bingham plastic; and
a compliant reservoir coupled to the inflatable portion and the access port, the compliant reservoir for automatically relaxing the constriction formed by the self-adjusting gastric band and allowing the large bolus to pass through the constriction by receiving the pseudoplastic fluid or the Bingham plastic from the inflatable portion.

23. A self-adjusting gastric band that adjusts to allow a large bolus of food to pass through a constriction in a patient's stomach formed by the self-adjusting gastric band, the self-adjusting gastric band comprising:
a plurality of closed constriction portions disposed about an esophageal-gastric junction for contacting and restricting the patient's stomach; and
a support structure attached to the plurality of closed constriction portions, the support structure including alternating ring portions and hinge portions, each ring portion contacting a different one of the plurality of closed constriction portions, each hinge portion separating adjacent ring portions, and configured to flex to allow the large bolus to pass through the constriction.

24. The self-adjusting gastric band of claim 23 wherein the plurality of closed constriction portions are filled with gas and wherein the plurality of closed constriction portions are each coated with a gas-impermeable substance.

25. The self-adjusting gastric band of claim 23 wherein the plurality of closed constriction portions are filled with a fluid.

26. The self-adjusting gastric band of claim 23 further comprising four constriction portions, four ring portions and four hinges.

27. A gas-impermeable self-adjusting gastric band that adjusts to allow a large bolus of food to pass through a constriction in a patient's stomach formed by the gas-impermeable self-adjusting gastric band, the gas-impermeable self-adjusting gastric band comprising:
a gas-impermeable inflatable portion disposable about an esophageal-gastric junction of the patient;
a gas-impermeable access port fluidly coupled to the inflatable portion to fill and drain the gas-impermeable inflatable portion; and
a gas-impermeable compliant reservoir fluidly coupled to the gas-impermeable inflatable portion and the gas-impermeable access port, the gas-impermeable compliant reservoir for automatically relaxing the constriction formed by the self-adjusting gastric band and allowing the large bolus to pass through the constriction by receiving gas from the gas-impermeable inflatable portion.

28. The gas-impermeable self-adjusting gastric band of claim 27 wherein the gas-impermeable inflatable portion, the gas-impermeable access port and the gas-impermeable compliant reservoir are each coated with a gas-impermeable substance.

29. The gas-impermeable self-adjusting gastric band of claim 27 wherein at least one of the gas-impermeable inflatable portion, the gas-impermeable access port and the gas-impermeable compliant reservoir is constructed out of silicone rubber.

30. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
a reservoir fluidly coupled to the inflatable portion and the access port, the compliant reservoir for automatically relaxing the constriction formed by the self-adjusting gastric band and allowing the large bolus to pass through the constriction by receiving both fluid and gas from the inflatable portion; and
an encapsulated gas-filled member configured to move from the inflatable portion to the reservoir in response to a pressure created by the large bolus passing through the constriction, the encapsulated gas-filled member further configured to move from the reservoir to the inflatable portion in response to a reduction of pressure when the large bolus has passed through the constriction.

31. The self-adjusting gastric band of claim 30 wherein the encapsulated gas-filled member is a balloon coated with a gas-impermeable coating.

32. The self-adjusting gastric band of claim 30 wherein the encapsulated gas-filled member is a closed-cell foam coated with a gas-impermeable coating.

33. The self-adjusting gastric band of claim 30 wherein the reservoir is compliant.

34. The self-adjusting gastric band of claim 30 wherein the reservoir is rigid.

35. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
an access port fluidly coupled to the inflatable portion to fill and drain the inflatable portion;
a reservoir fluidly coupled to the inflatable portion and the access port, the compliant reservoir for automatically relaxing the constriction formed by the self-adjusting gastric band and allowing the large bolus to pass through the constriction by receiving fluid from the inflatable portion; and
a gas spring located within the reservoir, the gas spring having a housing and a piston, the gas spring configured to move the piston within the housing in a direction away from the inflatable portion and compress a gas sealed between the inside walls of the housing and the piston in response to a pressure created by the large bolus.

36. The self-adjusting gastric band of claim 35 wherein the gas spring is configured to move the piston toward the inflatable portion and decompress the gas in response to a reduction of pressure caused by the large bolus passing through the constriction.

37. The self-adjusting gastric band of claim 36 further comprising a pin configured to stop the movement of the piston in response to the reduction of pressure caused by the large bolus passing through the constriction.

38. The self-adjusting gastric band of claim 35 wherein the outer surface of the housing comprises alternating curved portions and flat portions, the curved portions for contacting an interior surface of the reservoir and holding the housing in place, the flat portions for allowing the fluid to pass through the reservoir between the interior surface of the reservoir and the outer surface of the housing.

39. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient; and
an access port fluidly coupled to the inflatable portion to fill and drain the inflatable portion, the access port further configured to be a compliant member for automatically relaxing the constriction formed by the self-adjusting gastric band and allowing the large bolus to pass through the constriction by receiving fluid from the inflatable portion.

40. The self-adjusting gastric band of claim 39 wherein the access port further comprises a movable membrane which increases the fluid volume of the access port in response to a pressure induced by the large bolus passing through the constriction.

41. The self-adjusting gastric band of claim 39 wherein the access port further comprises a permeable membrane having openings configured to allow fluid to pass through while preventing a tip of a needle from passing through.

42. The self-adjusting gastric band of claim 39 wherein the access port further comprises a spring.

43. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
an access port fluidly coupled to the inflatable portion to fill and drain the inflatable portion;
a compliant reservoir fluidly coupled to the inflatable portion and the access port, the compliant reservoir for automatically relaxing the constriction formed by the self-adjusting gastric band and allowing the large bolus to pass through the constriction by receiving fluid from the inflatable portion; and
a restriction device for controlling a fluid flow rate between the inflatable portion and the compliant reservoir, the restriction device configured to temporarily increase the band-side pressure when a large bolus passes through the constriction.

44. The self-adjusting gastric band of claim 43 wherein the restriction device is a one-way valve, a pressure relief valve, an orifice, a turbulence controller, a sponge, or an environment adapting device.

45. The self-adjusting gastric band of claim 43 wherein the restriction device is electronically controlled, modulated with an external adjustment system or a passive restriction device configured to be independent from being controlled by an external tool.

46. The self-adjusting gastric band of claim 43 wherein the restriction device is an orifice having an inner diameter which limits the flow rate of the fluid in both directions.

47. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient for automatically relaxing the constriction formed by the self-adjusting gastric band and allowing the large bolus to pass through the constriction, the inflatable portion having a first end and a separate second end; and
a ring about the inflatable portion, the ring constructed out of metal and having a spring rate that provides a substantially constant force on the esophageal-gastric junction of the patient where the inflatable portion is disposed, the ring further configured to flex open to allowing the large bolus to pass through the constriction.

48. The self-adjusting gastric band of claim 47 further comprising a tether for connecting the first end and the second end.

49. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient, the inflatable portion having a first component for restricting and stimulating the esophageal-gastric junction, and a second component for dilation prevention, the second component attached to the first component in an orad direction, the second component increasing in diameter in the orad direction; and
a ring about the inflatable portion, the ring having a first component corresponding with the first component of the inflatable portion and a second component corresponding with the second component of the inflatable portion, the second component of the ring attached to the first component of the ring in an orad direction, the second component increasing in diameter in the orad direction.

50. The self-adjusting gastric band of claim 49 further comprising:
an access port fluidly coupled to the inflatable portion to fill and drain the inflatable portion; and
a compliant reservoir fluidly coupled to the inflatable portion and the access port, the compliant reservoir for automatically relaxing the constriction formed by the self-adjusting gastric band and allowing the large bolus to pass through the constriction by receiving fluid from the inflatable portion.

51. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient, the inflatable portion independent from a ring portion and structural support for the inflatable portion solely dependent on the inflatable portion;
an access port fluidly coupled to the inflatable portion to fill and drain the inflatable portion; and
a compliant reservoir fluidly coupled to the inflatable portion and the access port, the compliant reservoir for automatically relaxing the constriction formed by the self-adjusting gastric band and allowing the large bolus to pass through the constriction by receiving fluid from the inflatable portion.

52. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
a ring attached to the inflatable portion, the ring having a plurality of indented portions along a length of the ring;
an access port fluidly coupled to the inflatable portion to fill and drain the inflatable portion; and
a compliant reservoir fluidly coupled to the inflatable portion and the access port, the compliant reservoir for automatically relaxing the constriction formed by the self-adjusting gastric band and allowing the large bolus to pass through the constriction by receiving fluid from the inflatable portion.

53. The self-adjusting gastric band of claim 52 wherein the indented portions are holes which extend through the entire depth of the ring.

54. The self-adjusting gastric band of claim 52 wherein each of the indented portions include two tapered surfaces.

55. A vacuum device configured to assist a patient in transferring fluid from a gastric band to a fluidly coupled reservoir to allow a large bolus of food to pass through a constriction in a patient's stomach formed by the gastric band, the vacuum device comprising:
a body having a suction-creating component; and
a tip attached to the body, the tip configured to have a diameter substantially equal to a diameter of the reservoir, the tip configured to contact an area on an outer surface of the patient's body proximate to the location of the reservoir and form a seal on the contacted area to pull on skin of the patient and a subcutaneous fat layer located below the skin to induce fluid to flow from the gastric band to the reservoir.
